(19) United States
(12) Patent Application Publication
(10) Pub. No.: US 2008/0087293 A1
(43) Pub. Date: Apr. 17, 2008

(54) Hair treatment application system

(75) Inventors: Robert Wayne Glenn, Virgina Water (GB); Paul James Smith, Twickenham (GB)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company

(21) Appl. No.: 11/634,648

(22) Filed: Dec. 6, 2006

(30) Foreign Application Priority Data
Oct. 12, 2006 (EP) 06122213.9

(51) Int. Cl. A61K 9/70; A61K 8/18

(52) U.S. Cl. 132/210; 424/443; 424/70.2

(57) ABSTRACT

Disclosed herein is a hair treatment application system (10) and methods to use the same. The hair treatment application system (10) comprises the combination of at least one absorbent substrate (30) having specific median pore radius of from about 300 microns to about 3,000 microns and of one or more hair treatment composition (15), each having a viscosity of from about 3,00 cPs to about 150,000 cPs. The hair treatment application system (10) according to the invention allows for easy, non-messy and precise application of hair treatment compositions (15) in particular of highlighting compositions.

### FIELD OF THE INVENTION

The present invention relates to application devices which allow for quick, easy and non messy targeted application of cosmetic compositions to a variety of substrates such as keratinous fibres, teeth, finger nails and textiles. The present invention is especially intended for the application of hair treatment compositions to hair.

### BACKGROUND OF THE INVENTION

Self applied hair treatment compositions whether they are conditioning or styling compositions, sunscreen protective compositions, high and low lighting, colouring, bleaching, perming or so called special ‘party’ effects compositions are very desirable for the consumer. Among these hair treatment applications, the specific strand targeting of hair treatment compositions for example such as highlighting or colour-streaks is perceived by consumers as an ideal entry route into the hair colouring arena as it provides an opportunity to alter only a portion of their hair. Thus, it is considered as a low risk and allows the consumers the opportunity to familiarize themselves with the techniques and optimize the resulting effect. Moreover, highlighting is also considered by consumers to provide the opportunity to produce various looks from subtle multi-tonal effects and natural colour variations to more bold and daring effects.

However, a number of technical problems are associated with using self-applied hair treatment compositions without assistance.

A first problem concerns the fact that certain applications, such as applications of dyeing, highlighting and perming compositions, are particularly difficult to be executed satisfactorily without assistance. These applications, in view of the irreversible effects they produce, must occur carefully and precisely.

Another technical problem is associated with the portion of hair where these hair treatments are executed. Highlighting and perming for instance are likely to be carried out on a single hair strand and thus must be performed in an accurate and exact way as the inappropriate application might modify the overall appearance of the look, particularly when the selected hair strands are those close to the user's face. In addition for applications at the back of the head, the user has to act and take decisions on which strands to treat based on a mirror image, easily losing the overall head perspective.

Another technical problem to be considered is that application of hair treatment composition may lead to some mess when those hair treatment compositions are self-applied by the consumer.

This drawback is further amplified, when the hair treatment compositions are unpleasant for consumer contact. This is the case for example with bleaching, colouring and perming compositions as those compositions can easily cause staining or bleaching if dripped onto home surfaces or the consumer's skin. Thus, the consumers whilst having to apply the hair treatment compositions accurately to the hair, have to simultaneously avoid dripping of the hair treatment composition.

In order to facilitate the application of these self-applied hair treatment compositions onto the hair, the manufacturers of such products typically provide the consumer with an applicator. Nevertheless, even when the hair treatment compositions are applied through an applicator, the applicator is often not easy to hold or use and requires considerable practice, patience and experience in order to achieve at least a satisfactory result.

These applicators range from cap and hook type devices, brushes and comb like devices, which are either supplied separately or are designed to be attached to the hair treatment composition bottle. None of these applicators is particularly satisfactory. The cap and hook method requires the consumer to place an apertured cap over the hair and then to select hair strands which are to be subjected to the hair treatment through the apertures with a hook. The brushes, wand and comb devices require the consumer to load the device with the treatment composition and then apply along the length of a selected hair strand, reloading as required. The comb devices typically have a chamber to load a predetermined quantity of composition. The comb and bottle devices require simultaneous squeezing of the bottle to release the treatment composition whilst combing the hair. These applicators require the consumer to personally determine the width of the hair strand to be treated and to separate it from the rest of the hair. Alternatively, only a predetermined strand width selection can be treated and strand separation is not required, thus precluding any variation. Finally, it is highly undesirable that any contamination of the untreated remaining hair with the self-applied hair treatment composition occurs.

Thus, these applicators do not provide the consumer with the easy and quick hair treatment application that is desired. Particularly problematic are the difficulties to apply product to the root area, either to cover newly grown grey hair or to adjust the colour or bleach the virgin hair. The overall characteristic of the hair at the root line is different and due to its close vicinity to the scalp, it is really difficult to handle. Exact placement at the root line is difficult to achieve and contamination of neighbouring hair areas is a common problem.

Furthermore the applicators also do not allow for single root to tip application of the composition unless on very short hair, requiring the consumer to stop, to reload and then to start the application again by reapplying at mid length.

Whilst some consumers may be reasonably satisfied with the end result achieved with the currently available products, due to the time and effort required many consumers still migrate to the services offered by professional hair salon stylists in order to attain the desired result.

The use of hair salon stylists allows the consumer to achieve personal customization of the end look. The salon professionals have a number of applicators and techniques at their disposal which together with training and years of experience allow for the provision of very varied results. However, even within the hair salon environment, the specific application on hair strands of a treatment composition is also a lengthy process. In order to accurately apply the composition and minimize messiness, the consumer is required to regularly spend a number of hours at the salon in order to complete the process. Because of the long time and effort employed by the stylists to achieve the expected end results a very high revenue is demanded for their services. This is not available for all consumers' budgets and thus, home application highlighting products are also highly desirable in terms of cost in comparison with the professional hair stylists.

There is hence still a need to provide a hair treatment application system which can easily, quickly and precisely, deliver at home as well as at professional salons, hair treatment compositions particularly hair highlighting compositions.

Some attempts to address these problems are known in the art. Application articles composed of multiple layers of different materials have been described in US 2002/0142027 and US 2005/0079192. US 2002/0142027 discloses an article, which comprises a cavity to receive part of the human body. The cavity has a cover defined as to have a composite structure of at least two layers, one of which must be permeable to a thin solvent such water. An adhesive matrix is situated between those two layers and comprises the active substance to be applied. US 2005/0079192 relates to an article impregnated with a cosmetic composition. The article is composed of an absorbent structure and a contact structure. The contact structure comes into contact with a moist surface and has the characteristics of being on one side permeable to the water present on the moist surface and of being hydrophobic on the side adjacent to the absorbent structure.

Sponges able to deliver cosmetic compositions are disclosed in DE 10259016. Such sponges have absorbance capacity and retention capacity so as to deliver all the absorbed composition by applying only minimal pressures.

Diverse are the attempts proposed by the prior art to solve the technical problem of precisely delivering a hair treatment composition on a defined strand with applicator devices avoiding dripping of the hair treatment composition and/or independently on the orientation of the applicator device thus allowing self-application.

U.S. Pat. No. 6,626,599 discloses an applicator comprising a cylinder-shaped reservoir containing a hair treatment composition, wherein said reservoir has a lateral opening where an elastic deformable material is lodged. Through that opening the hair strand is set into communication with the composition contained in the reservoir.

JP 1991178630 discloses a hair cosmetic applicator characterized by two hinged plates having attached two bags comprising similar or different compositions. Each bag contains a hole from which the composition is released by opening-closing the hinged plates.

Thus, it remains the need for an applicator device capable of accommodating cosmetic compositions, particularly hair treatment compositions, which allows for their easy application on different receiving surfaces without uncontrolled dripping. In particular, the applicator device should allow the application of hair treatment compositions on hair, particularly on single hair strands or on hair at the root line and should also allow easy application on portions of the head difficult to achieve such as the back of the head.

It has now been surprisingly found that a hair treatment application system, as defined herein after, can significantly improve the highlighting results through a simple, intuitive and non-messy application.

### SUMMARY OF THE INVENTION

According to the invention, a hair treatment application system (10) is provided, comprising at least one absorbent substrate (30) and one or more hair treatment compositions (15), wherein said at least one absorbent substrate (30) has a median pore radius of from 300 microns to 3,000 microns and wherein said one or more hair treatment compositions each has a viscosity of from 3,000 cPs to 150,000 cPs. Said viscosity is measured before said one or more hair treatment compositions (15) are applied to said at least one absorbent substrate (30).

Said at least one absorbent substrate (30) of said hair treatment applicator system (10) has preferably an absorption capacity for said one or more hair treatment composition (15) of from 10 to 80 grams of liquid per gram of absorbent substrate.

The present invention further relates to methods of treating the hair with said hair treatment application system (10) and kit-of-parts comprising said hair treatment application system (10).

### BRIEF DESCRIPTION OF THE DRAWINGS

FIG. 1 shows said hair treatment application system (10) and the method of use thereof. On the left side of FIG. 1, the hair treatment application system (10) is held with one hand, then as shown in the right side it is contacted with the hair strand (11) and swiped from the root-line to the tips to apply the hair treatment composition (15).

FIG. 2 shows an embodiment of the hair treatment applicator system (10) according to the invention comprising an absorbent substrate (30) attached to a plate (22), wherein said plate (22) is connected to a handle (21) for ease of use.

FIG. 3 shows a hair treatment application system (10) according to the invention comprising a hair treatment composition (15) applied onto the inner surface (32) of an absorbent substrate (30). The absorbent substrate (30) is attached on its outer surface (31) to the internal surfaces (52) of two plates (22). The plates are thus connected via said absorbent substrate (30).

FIG. 4 shows a hair treatment application system (10) according to the invention comprising two absorbent substrates (30) attached via their outer surfaces (31) to a flexible pliable support comprising a container (40). A hair treatment composition (15) is applied onto one of the two absorbent substrates' (30) inner surface (32) and into the container (40).

FIG. 5 shows a hair treatment application system (10) comprising two absorbent substrates (30) attached to two plates (22) connected via a hinge (50), wherein one absorbent substrate (30) comprises a hair treatment composition (15) (left). By applying a force onto the external surfaces (51) of the hinged plates (22) the absorbent substrates (30) come into contact.

FIG. 6 shows the loading of a hair treatment composition (15) on the inner surface (32) of an absorbent substrate (30) of a hair treatment application system (10).

### DETAILED DESCRIPTION OF THE INVENTION

For the purpose of this invention, the term hair refers to both living hair i.e. on a living body and to non-living hair i.e. in a wig, hairpiece or other aggregation of non-living keratinous fibre. Mammalian, preferably human hair is intended. However, wool, fur and other keratinous fibre may be suitable to be used with the hair treatment application system (10) according to the invention.

The term hair strand, for the purpose of this invention, refers to at least two keratinous fibres, especially hair, in particular human hair and it should be construed as hair bundle.

As used herein, the term “applied” when referring to a hair treatment composition is to encompass coated, loaded, absorbed, adsorbed and adhered.

While the specification concludes with claims, which particularly point out and distinctly claim the invention, it is believed the present invention will be better understood from the following description.

In examining how to solve the technical problem discussed hereinabove, the present inventor has determined that the delivery of thin fluids from conventional absorbent structures to flat surfaces, i.e., baby wipes, bathroom wipes, kitchen wipes, floor wipes, kitchen cleaning sponges or facial wipes are not applicable for application to hair. Unlike these applications where the thin fluid can be deposited and then smeared across a predominantly flat surface and thereby contained, for delivery to hair, in particular to hair strands, which are cylindrical in shape and without gravimetric confinement to a flat surface, the use of a low viscosity fluid conventional absorbent structures leads to significant consumer undesired dripping and messiness.

The present inventor has now surprisingly identified that the specific selection of the viscosity of the hair treatment compositions of from about 3,000 cPs to about 150,000 cPs in combination with the specific selection of the median pore radius of absorbent substrates of from about 300 microns to about 3,000 microns are critical in order to deliver the desired technical effect.

If the viscosity of the hair treatment composition is too high, it is not easily loaded onto the absorbent substrate and the delivery from the absorbent substrate is too difficult, moreover inter-mixing of two or more differing hair treatment compositions, which is desirable for reactive hair treatment applications such as oxidative dyeing and bleaching including highlighting, if required, can be prohibited. On the other hand if the viscosity of the hair treatment composition is too low the loading may be inefficient causing messiness as the hair treatment composition is not sufficiently retained within the absorbent substrate, causing undesirable dripping and messiness.

As demonstrated hereinafter by the technical experimental data, the minimum viscosity of the hair treatment compositions, measured according to the test method described hereafter, is of at least 3,000 cPs. Carbopol 956 solutions (available form Noveon) prepared at various concentrations to afford a comprehensive range of viscosities were tested with diverse types of absorbent substrates having a wide range of median pore radii. Each of the Carbopol 956 solutions was loaded into the chosen absorbent substrates. The absorbent substrates were then compressed as described in the test method herein after. Each experiment was repeated three times and the results were evaluated using the Student t-test (∝=0.05) and statistically different averages are denoted by different letters in square brackets for each absorbent substrate. The average percentage of the loaded Carbopol 956 solution that had dripped out from the absorbent substrate under compression and the corresponding viscosities are shown in Table 1.

TABLE 1
Effect of viscosity changes versus median pore radius of the absorbent substrate.

| Absorbent | BBA Fiberweb | | Libeltex | | Recticel Bulpren | |
|---|---|---|---|---|---|---|
| substrates | Tenotex P101 | | 01-766 DI-8 | | S28280 | |
| Pore Radii | 75 microns | | 550 microns | | 1,400 microns | |
| Viscosity [cPs] | Average | Std Dev. | Average | Std Dev. | Average | Std Dev. |
| 1000 | 29.93 [A] | 1.16 | 32.95 [A] | 1.50 | 10.08 [A] | 1.48 |
| 1500 | 26.26 [B] | 3.60 | 30.29 [B] | 1.24 | 7.93 [B] | 1.1.88 |
| 2000 | 12.96 [C] | 2.42 | 17.66 [C] | 0.92 | 3.59 [C] | 1.60 |
| 3000 | 0 [D] | 0 | 8.92 [D] | 0.74 | 0 [D] | 0 |
| 3500 | 0 [D] | 0 | 0 [E] | 0 | 0 [D] | 0 |
| 5000 | 0 [D] | 0 | 0 [E] | 0 | 0 [D] | 0 |
| 7500 | 0 [D] | 0 | 0 [E] | 0 | 0 [D] | 0 |
| 10000 | 0 [D] | 0 | 0 [E] | 0 | 0 [D] | 0 |

These results clearly illustrate that as the viscosity increases the percentage of Carbopol 956 solution that drips out from the absorbent substrate significantly decreases. Surprisingly, it has been found that, independently from the absorbent substrate chosen, dripping was substantially absent when the absorbent substrates were loaded with a Carbopol 956 solution having viscosity of at least about 3,000 cPs

To further support the above discussed experimental data on the hair treatment composition according to the invention, a consumer testing with 12 panellists was also undertaken. Each panellist was given an absorbent substrate (Libeltex 01-766 DI-8, median pore radius 550 microns) to test and to evaluate its messiness and dripping The absorbent substrate was mounted on one side of a plate as described herein after in the test methods section. Two of so prepared plates were hinged together so as to form a clip with the absorbent substrates facing one the other. One absorbent substrate per clip was loaded with solutions of Carbopol 956 having a viscosity of 1,000 cPs. Other clips were prepared with Carbopol 956 solutions having viscosities of 2,500 cPs and 5,000 cPs.

The results were evaluated with the Fisher's LSD statistical test (∝=0.05), revealing the average grade for both the Carbopol 956 solutions at viscosity of 1,000 and 2,500 cPs viscosity fluids are statistical identical for both questions and that the one at 5,000 cPs is significantly different for each question and rated as being less messy. These results, shown in table 2, support the technical experimental data that the viscosity required to avoid messiness is in the range between 2,500 cPs to 5,000 cPs. Statistically different averages are denoted by different letters in square brackets.

TABLE 2
Consumer testing for messiness.

| Question | Considering the sample you have just used, |
|---|---|
| | how would you rate its mess? |
| Grade | Scale: 0–8 |
| | 0 = Not Messy at All |
| | 4 = Somewhat Messy |
| | 8 = Extremely Messy |
| Viscosity [cPs] | Average of All Grades |
| 1000 | 3.83 [B] |
| 2500 | 3.92 [B] |
| 5000 | 2.00 [A] |

The present inventors have also surprisingly found that a very specific type of absorbent substrate is required. Specifically, the absorbent substrates of the present invention are selected such that they posses a median pore radii that can accommodate one or more hair treatment compositions having a minimum viscosity of at least 3,000 cPs. The porous nature of absorbent substrates is accurately described by the median pore radius. This is conventionally established via the TRI Autoporosimeter® methodology for samples with median pore radii less than or equal to 1000 microns and by optical imaging for samples with radii greater than 1000 microns as defined herein after. The effect of median pore radius of absorbent substrates useful within the scope of the present invention was determined using a gravimetric fluid loading as described herein after in the test method section. This method represents at the best how a consumer may load absorbent substrates with hair treatment compositions. The absorbent substrates chosen were BBA Fiberweb Tenotex P101 (median pore radius of 75 microns); Freudenberg AL 1060 (300 microns); Libeltex 01-766 DI-8 (550 microns); PGI FB-215 (700 microns) and Recticel Bulpren S28280 (1,400 microns). Each absorbent substrate was loaded with Carbopol 956 solutions having a viscosity of 3,000 cPs, 5,000 cPs and 10,000 cPs, respectively. Each experiment was repeated three times and results were evaluated using the Student t-test (∝=0.05) and statistically different averages are denoted by different letters in square brackets. The average amount (in grams) of Carbopol 956 solution loaded into each absorbent substrate at the corresponding viscosity is shown in Table 3.

TABLE 3
Effect of the minimum median pore radius of the absorbent substrate on loading.

| Absorbent | BBA Fiberweb | | Freudenberg AL | | | | | | Recticel Bulpren | |
|---|---|---|---|---|---|---|---|---|---|---|
| substrate | Tenotex P101 | | 1060 | | Libeltex 01-766 DI-8 | | PGI FB-215 | | S28280 | |
| Pore Radii | 75 microns | | 300 microns | | 550 microns | | 700 microns | | 1,400 microns | |
| Viscosity [cPs] | Average | Std Dev. | Average | Std Dev. | Average | Std Dev. | Average | Std Dev. | Average | Std Dev. |
| 3000 | 1.25 [E] | 0.08 | 6.15 [D] | 0.92 | 30.24 [B] | 2.62 | 37.54 [A] | 0.85 | 24.33 [C] | 0.48 |
| 5000 | 0.70 [E] | 0.03 | 5.41 [D] | 0.53 | 24.86 [C] | 0.56 | 33.61 [A] | 1.68 | 27.38 [B] | 2.41 |
| 10000 | 0.40 [E] | 0.04 | 2.82 [D] | 0.30 | 17.50 [C] | 0.61 | 22.38 [A] | 0.98 | 21.24 [B] | 0.28 |

The results illustrate that by increasing the median pore radius of the absorbent substrate, the amount of Carbopol 956 solution which can be loaded into each absorbent substrate also increases. Furthermore, the results indicates that Carbopol 956 solutions having minimum viscosity of 3,000 cPs could not significantly be loaded when the absorbent substrate has a median pore radius of less that 300 microns, such in the case of BBA Fiberweb Tenotex P101 which has a median pore radius of 75 microns.

Thus, for the purpose of the present invention, the median pore radius of the absorbent substrate is of at least about 300 microns. Below this minimum median pore radius, hair treatment compositions of viscosity of at least about 3,000 cPs cannot be accommodated for uses in the intended consumer applications of the present invention.

The maximum median pore radius of the absorbent substrates selected herein was determined to be the maximum radius of an open cell foam that is feasible to manufacture, that is commercially available and that is still capable of holding a shape while it is used for the intended consumer applications of the present invention. The maximum median pore radius open cell foam is provided by Recticel and has a median pore radius of 3,000 microns. Absorbent substrates having median pore radii higher than 3,000 microns have a tendency to lose their mechanical strength and thus are not employed within the scope of the present invention. Thus, the absorbent substrates according to the present invention have a median pore radius of from about 300 microns to about 3,000 microns, preferably from about 400 microns to about 2,500 microns, more preferably from about 450 microns to about 2,000 microns, even more preferably from about 500 microns to about 1,800 microns.

Hair treatment compositions have very diverse viscosities and presently commercially available products have viscosities up to 600,000 cPs. Within the scope of the present invention, the maximum viscosity of a hair treatment composition that can still be loaded on the absorbent substrates as selected above and delivered to the hair is 150,000 cPs. Two absorbent substrates representative of the above determined median pore radius range, Recticel Pottscorer 410 (1,400 microns) and Recticel Bulpren S28280 (2,200 micron), were chosen. These two absorbent substrates were then loaded with a hair treatment composition having viscosity of 100,000 cPs and three hair bundles were subsequently contacted with the absorbent substrates to coat the hair treatment composition on the hair. In both cases the absorbent substrates were able to provide an amount in grams of hair treatment composition per grams of hair that achieved highlights. These results are shown in table 4 below.

TABLE 4
Effect of the viscosity on highlighting performance

| | Recticel Bulpren | | Recticel | |
|---|---|---|---|---|
| Absorbent substrate | S28280 | | Pottscorer 410 | |
| Pore Radii | 1,400 microns | | 2,200 microns | |
| Mileage | Average | Std Dev. | Average | Std Dev. |
| Highlight 1st | 1.73 | 0.19 | 1.42 | 0.32 |
| Highlight 2nd | 1.50 | 0.30 | 0.68 | 0.30 |
| Highlight 3rd | 1.46 | 0.22 | 0.43 | 0.29 |

Although these results refer to a viscosity value of 100,000 cPs, the one skilled in the art of hair treatment compositions would find it easy to understand that hair treatment compositions having viscosity up to about 150,000 cPs could still be loaded on the absorbent substrates according to the invention and applied to the hair. Thus, the hair treatment compositions according to the present invention are defined to have a viscosity of from about 3,000 cPs to about 150,000 cPs, more preferably from about 5,000 cPs to about 125,000 cPs, more preferably from about 7,000 cPs to about 100,000 cPs, even more preferably from about 9,000 cPs to about 85,000 cPs, wherein said viscosity is measured before application into the absorbent substrates according to the test method described hereinafter.

In addition to the median pore radius, it has also been determined that selecting a defined absorbent capacity may also improve the application of the hair treatment composition onto the hair. The absorption capacities of the absorbent substrates of the present invention can be described by the theoretical maximum uptake of water in grams per gram of substrate (grams per gram). The absorption capacities of the absorbent substrates of the present invention are preferably in the range of from about 10 to about 80 grams of hair treatment composition per gram of absorbent substrate, preferably from about 15 to about 75 grams per gram, more preferably from about 20 to about 70 grams per gram, and even more preferably from about 25 to about 65 grams per gram of absorbent substrate as determined according to the calculation described herein after in the test method section.

The inventors have also determined that a selected calliper range of the absorbent substrate may also assist in delivering the required amount of hair treatment composition to the hair. The calliper is measured at the highest point on the absorbent substrate's surface conventionally via a micrometer according to the test method described herein after. The callipers of the absorbent substrates of the present invention are preferably in the range of from about 2 to about 20 mm, preferably from about 3 to about 17 mm, more preferably from about 4 to about 12 mm, and even more preferably from about 5 to about 10 mm.

Similarly, the selection of absorbent substrate materials which have a specific basis weight may also enhance the absorption capacity. Preferably the absorbent substrates of the present invention also have a basis weight of from about 20 to about 300 g/m², preferably from about 60 to about 250 g/m², more preferably from about 100 to about 200 g/m², according to the test method described herein after.

Another parameter which has also been identified as resulting in the delivery of improved dosage of the composition onto the hair and the prevention of dripping is the liquid permeability. Preferably, the absorbent substrate has a liquid permeability of from about 500 Darcy to about 5500 Darcy, preferably from about 800 Darcy to about 4500 Darcy and more preferably of from about 1000 Darcy to about 3000 Darcy as defined according to the test method herein below.

### 1. ABSORBENT SUBSTRATES

Suitable absorbent substrates for use in the present invention may be selected from non-wovens; wovens; porous foams and foam materials; porous plastics; flexible frits; meshes; and combinations thereof including recycled and composite materials having one or more plies of the same or different materials superimposed physically, joined together continuously (laminated), in a discontinuous pattern, or by bonding the external edges at discrete loci provided that the structures meet the functional requirements described hereinabove.

The absorbent substrates of the present invention are preferably selected from non-wovens and/or porous foams.

Non-woven materials are produced from fibers that may be staple or continuous filaments or be formed in situ and include a manufactured sheet, web or batt or directionally or randomly oriented fibers, bonded by friction, and/or cohesion and/or adhesion. Nonwoven webs and processes for making them may comprise three steps: fiber laying, precursor web formation, and fiber bonding. The fiber laying step may be comprised of the spunlaying, meltblowing, carding, airlaying, wetlaying and combinations thereof, of the fibers comprising the web onto a forming surface. The step of precursor web formation may prevent the fibers comprising the web from coming apart during the bonding step. Precursor web formation may be performed via a pre-bonding step, such as one that is chemical or mechanical in nature. The bonding step may then impart strength to the finished web. The bonding step may be comprised of subjecting the fibers comprising the web to hydroentanglement (HET), cold calendering, hot calendering, air through bonding, chemical bonding, needle punching, and combinations thereof. Suitable non-woven materials may be comprised of natural or synthetic fibers selected from acetate fibers; acrylic fibers; cellulose ester fibers; modacrylic fibers; polyamide fibers; polyester fibers; polyolefin fibers; polyvinyl alcohol fibers; rayon fibers; keratin fibers; cellulose fibers; silk fibers and combinations thereof. The non-wovens may be comprised of mono-component fibers, such as a polyolefin or polyester, or bi-component fibers, such as a sheath/core fiber or side by side fiber of polyethylene/polypropylene or polyethylene/polyester, or bi-constituent fibers comprised by a blend of two or more thermoplastic polymers.

The preferred non-woven substrates are selected from Carded, Air-laid, and Meltblown non-woven materials or composites. More preferably, the non-woven substrates of the present invention are selected from Carded webs produced by a carding machine with one of more different types of fibres. Even more preferably, the non-woven substrates of the present invention are selected from multi-layer or lofty web which are consolidated by through air bonding or needle-punching, often referred to as batting battings. Examples of suitable Carded non-wovens for use herein include; Libeltex Thermo-contact 01-766 DI-8; Libeltex Loftfill HC2; PGI FB-215; PGI FB-204B, PGI FB-185 and PGI FB-217.

Porous foams and foam materials are made from low density elastomers, plastics, and other materials with various porosities and may be selected from open cellular foams; flexible foams; rigid foams; and reticular foams and syntactic foams which can be fabricated into finished shapes using molding, casting, extrusion, pultrusion, machining, thermal forming, plastic welding, blow molding, rapid prototyping techniques, grinding and/or other specialized processes. The porous foams and foam materials may be composed of a variety of chemical systems including acrylonitrile-butadiene-styrene (ABS); acrylics; epoxy resins; fluoropolymers; isoprene-styrene (SIS) and styrene-butadiene-styrene (SBS); synthetic rubbers or elastomers based on a variety of systems such as silicone, polyurethane and neoprene; nitrile rubbers; plastics or elastomers formed from natural or plant-based raw materials such as natural rubber (polyisoprene) or vulcanized fibre; water-based and water-borne resins and latex materials. Chemical systems for porous foams and foam materials may include ethylene copolymer, expanded polyethylene, polycarbonate, polyester, polyether, polyetherimide, polyimide, polyolefin, polypropylene, polyurethane, phenolic, polyurea, and vinyl.

Porous plastics can be made from wide variety of materials including PolyTetraFluoroEthylene (PTFE), PolyEthylene (PE), PolyPropylene (PP), and Polyvinyldifluoride (PVDF). They are created by filling a mold with tiny plastic pellets, subjecting the mold to heat and pressure so the pellets bond where they touch. This part is then heated outside the mold; the part shrinks significantly during this step which strengthens it.

The porous foams are preferably polyurethane foams. Suitable examples of porous foams are available from Recticel International (Belgium) and include Sweepex S 31 CS/R, Bulpren S28280, Bulpren D32133, Filtren T23220, and Filtren TM 23133.

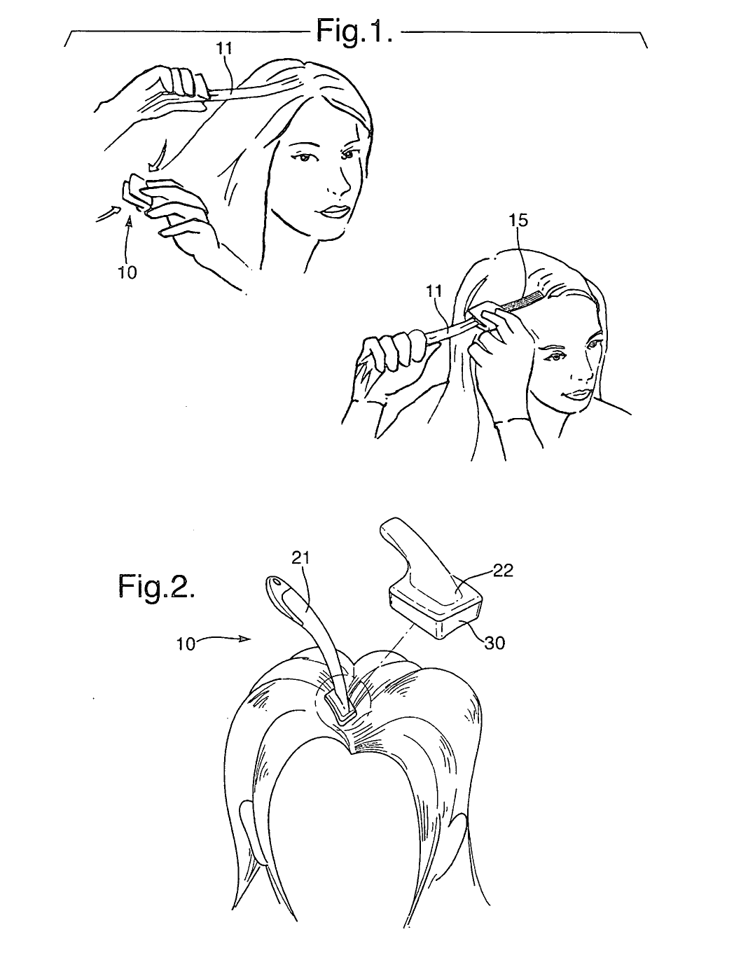

The absorbent substrates (30) of the present invention are typically provided such that they can easily fit in the hand of a user as shown in FIGS. 1 and 6 or an optional application tool described herein after and as shown in FIGS. 2 to 5. Any shape of the absorbent substrate (30) may be used such as circular, oblong or rectangular shapes. Each substrate has an outer (31) and an inner (32) surface. The outer surface (31) faces the user's hand and the inner surface (32) is the surface which is loaded with the hair treatment composition (15) and comes into contact with the hair.

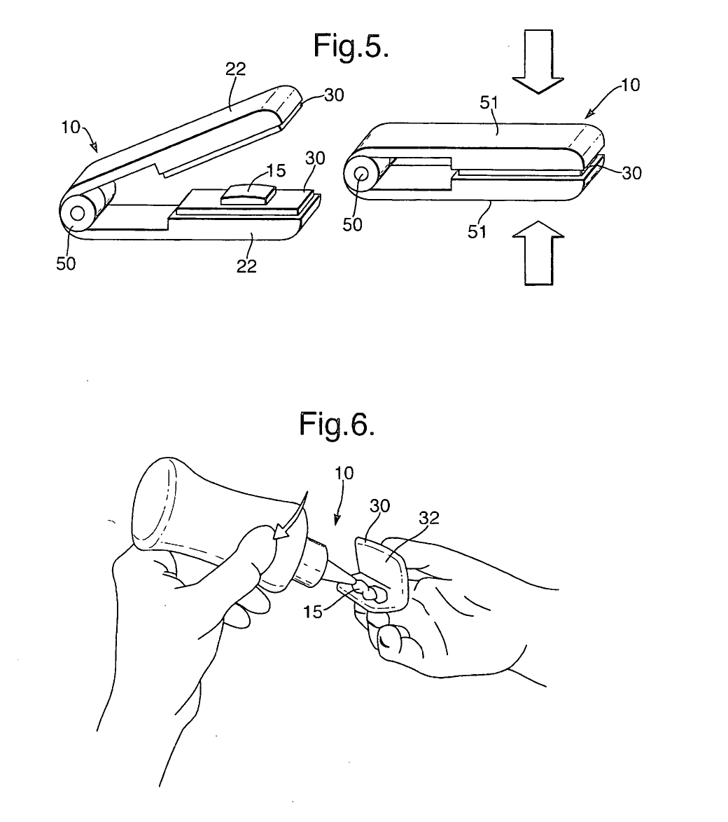

The hair treatment application system (10) of the present invention comprises at least one absorbent substrate (30) as shown in FIGS. 1, 2, 3 and 6, preferably at least two absorbent substrates (30) as shown in FIGS. 4 and 5.

Finally, the absorbent substrates (30) according to the invention are preferably substantially inert to the hair treatment compositions (15).

### 2. HAIR TREATMENT COMPOSITION

As discussed hereinabove, the inventors have surprisingly determined that the viscosity of the hair treatment composition (15) must be selected in combination with the median pore radius of the absorbent substrate (30) in order to provide the expected technical effect. Accordingly it has been surprisingly found that for effective loading and retention of the hair treatment composition (15) into the absorbent substrate (30), said one or more hair treatment compositions (15) each has a viscosity of from about 3,000 cPs to about 150,000 cPs, preferably from about 5,000 cPs to about 125,000 cPs, more preferably from 7,000 cPs to about 100,000 cPs, even more preferably from about 9,000 cPs to about 85,000.

The viscosity of the hair treatment composition (15) is measured before the hair treatment composition (15) is loaded into the absorbent substrate (30). The viscosity is measured for liquid hair treatment compositions (15) only. The term liquid hair treatment composition as used therein means a liquid form of a hair treatment composition such as paste, gel, solutions water-in-oil emulsions or other suitable forms, provided that they are in the form of a liquid when delivered to head and that have viscosity within the range claimed herein. Preferably the compositions are applied onto the absorbent substrates (30) in the form of gels which provide good adhering properties to the absorbent substrate (30). Hydrogels are especially preferred as they provide a source of water that facilitates diffusion and absorption of the hair treatment compositions (15) within the absorbent substrate (30).

According to the present invention, there are a number of ways to provide the desired hair treatment composition mixture in the absorbent substrate. Consequently, the term before as used herein has the following meaning for these executions:

When two identical hair treatment compositions (15) are loaded independently into at least one absorbent substrate (30) and then mixed within said at least one absorbent substrate (30), the viscosity of the hair treatment composition is measured before loading one of said hair treatment composition (15) onto said at least one absorbent substrate (30).

When two different hair treatment compositions (15) are premixed before loading into at least one absorbent substrate (30), the viscosity is measured on the resulting mixed hair treatment composition (15) before the premixed hair treatment composition is loaded into the absorbent substrate.

When two (or more) different hair treatment compositions (15) are independently loaded into at least one absorbent substrate (30) and then mixed within said at least one absorbent substrate (30), the viscosity of each single hair treatment compositions (15) is measured before loading into said at least one absorbent substrate (30). For embodiments involving the loading of two different compositions into the absorbent substrate, which are subsequently mixed within the absorbent substrate, it is preferred that the viscosity of the resulting mixed hair treatment composition has a viscosity of from 3,000 cPs to 150,000 cPs. For such embodiments, the viscosity of the resultant mixed hair treatment compositions is determined by mixing the two (or more) hair treatment compositions prior to loading into the absorbent substrate as described hereinafter in the test method section. This specific embodiment is exemplified in examples A and B here after.

When one of the hair treatment compositions (15) is a powder, then the viscosity of the hair treatment composition (15) resulting from the mixing of said powder with another liquid hair treatment composition (15) is measured.

It is also believed that for some hair treatment compositions (15) it is desirable to have a high degree of pseudo-plasticity to facilitate non-messiness, good performance, and a precise application of the hair treatment composition (15) only within the selected hair strand avoiding cross-contamination to the rest of the hair.

### 3. HAIR TREATMENT COMPOSITIONS APPLIED TO THE ABSORBENT SUBSTRATES

Any hair treatment composition (15) characterized by having the viscosity within the ranges selected herein can be employed in the underlying invention and can be loaded in the selected absorbent substrates (30) to achieve the technical effect described above. Examples of hair treatment compositions (15) that may be applied to the absorbent substrate(s) (30) are discussed below. Suitable hair treatment compositions (15) include shampoos, conditioners, styling compositions, hair colourants, bleaches, and highlighting compositions.

Accordingly, the compositions may comprise components known, conventionally used, or otherwise effective for use in hair treatment compositions (15) particularly oxidative bleaching and dye compositions which include but are not limited to: developer dye compounds; coupler dye compounds; direct dyes; oxidizing agents; thickeners; chelants; pH modifiers and buffering agents; carbonate ion sources and radical scavenger systems; anionic, cationic, nonionic, amphoteric or zwitterionic surfactants, or mixtures thereof; anionic, cationic, nonionic, amphoteric or zwitterionic polymers, or mixtures thereof; fragrances; dispersing agents; peroxide stabilizing agents; proteins and derivatives thereof, plant materials (e.g. aloe, chamomile and henna extracts); silicones (volatile or non-volatile, modified or non-modified), film-forming agents, ceramides, preserving agents, colour indicators and opacifiers. Some adjuvants referred to above, but not specifically described below, which are suitable are listed in the International Cosmetics Ingredient Dictionary and Handbook, (8th ed.; The Cosmetics, Toiletry, and Fragrance Association). Particularly, vol. 2, sections 3 (Chemical Classes) and 4 (Functions) are useful in identifying specific adjuvants to achieve a particular purpose or multipurpose.

A. Solvents

The medium suitable for dyeing may be selected from water or a mixture of water and at least one organic solvent to dissolve the compounds that would not typically be sufficiently soluble in water. Suitable organic solvents for use herein include, but are not limited to: C1 to C4 lower alkanols (e.g., ethanol, propanol, isopropanol), aromatic alcohols (e.g. benzyl alcohol and phenoxyethanol); polyols and polyol ethers (e.g., carbitols, 2-butoxyethanol, propylene glycol, propylene glycol monomethyl ether, diethylene glycol monoethyl ether, monomethyl ether, hexylene glycol, glycerol, ethoxy glycol), and propylene carbonate. Organic solvents are typically present in an amount ranging from about 1% to about 30%, by weight, of the composition. Preferred solvents are water, ethanol, propanol, isopropanol, glycerol, 1,2-propylene glycol, hexylene glycol, ethoxy diglycol, and mixtures thereof.

B. Oxidative Dye Compounds

The compositions of the present invention may include oxidative dye compounds in the form of primary intermediates or couplers. The compounds suitable for use in the inventive compositions (including those optionally added), in so far as they are bases, may be used as free bases or in the form of their physiologically compatible salts with organic or inorganic acids, such as hydrochloric, hydrobromic, citric, acetic, lactic, succinic, tartaric, or sulfuric acids, or, in so far as they have aromatic hydroxyl groups, in the form of their salts with bases, such as alkali phenolates.

Optional couplers are typically present in an amount such that in aggregate the concentration of couplers and the present discrete particle aggregates and/or agglomerates in the composition ranges from about 0.002% to about 10%, preferably from about 0.01% to about 5%, by weight, of the hair dyeing composition. Optional primary intermediates are present in an effective dyeing concentration, typically an amount from about 0.001% to about 10%, preferably from about 0.01% to about 5%, by weight, of the hair dyeing composition. The total amount of dye compounds in the hair dyeing compositions of this invention will typically range from about 0.002% to about 20%, preferably from about 0.04% to about 10%, more preferably from about 0.1% to about 7%, by weight, of the hair dyeing composition.

These compounds are well known in the art, and include aromatic diamines, aminophenols, aromaticdiols and their derivatives (a representative but not exhaustive list of oxidation dye precursor can be found in Sagarin, “Cosmetic Science and Technology”, “Interscience, Special Edn. Vol. 2 pages 308 to 310). It is to be understood that the precursors detailed below are only by way of example and are not intended to limit the compositions and processes herein. These are: 1,7-Dihydroxynaphthalene (1,7-NAPHTHALENEDIOL), 1,3-Diaminobenzene (m-PHENYLENEDIAMINE), 1-Methyl-2,5-diaminobenzene (TOLUENE-2,5-DIAMINE), 1,4-Diaminobenzene (p-PHENYLENEDIAMINE), 1,3-Dihydroxybenzene (RESORCINOL), 1,3-Dihydroxy-4-chlorobenzene, (4-CHLORORESORCINOL), 1-Hydroxy-2-aminobenzene, (o-AMINOPHENOL), 1-Hydroxy-3-aminobenzene (m-AMINOPHENOL), 1-Hydroxy-4-amino-benzene (p-AMINOPHENOL), 1-Hydroxynaphthalene (1-NAPHTHOL), 1,5-Dihydroxynaphthalene (1,5-NAPHTHALENEDIOL), 2,7-dihydroxynaphthalene (2,7-NAPHTHELENEDIOL) 1-Hydroxy-2,4-diaminobenzene (4-DIAMINOPHENOL), 1,4-Dihydroxybenzene (HYDROQUINONE), 1-Hydroxy-4-methylaminobenzene (p-METHYLAMINOPHENOL), 6-Hydroxybenzo-morpholine (HYDROXYBENZOMORPHOLINE), 1-Methyl-2-hydroxy-4-aminobenzene (4-AMINO-2-HYDROXY-TOLUENE), 3,4-Diaminobenzoic acid (3,4-DIAMINOBENZOIC ACID), 1-Methyl-2-hydroxy-4-(2′-hydroxyethyl)aminobenzene (2-METHYL-5-HYDROXY-ETHYLAMINO-PHENOL), 1,2,4-Trihydroxybenzene (1,2,4-TRIHYDROXYBENZENE), 1-Phenol-3-methylpyrazol-5-on (PHENYLMETHYLPYRAZOLONE), 1-(2′-Hydroxyethyloxy)-2,4-diaminobenzene (2,4-DIAMINOPHENOXY-ETHANOL HCL), 1-Hydroxy-3-amino-2,4-dichlorobenzene (3-AMINO-2,4-DICHLORO-PHENOL), 1,3-Dihydroxy-2-methylbenzene (2-METHYLRESORCINOL), 1-Amino-4-bis-(2′-hydroxyethyl)aminobenzene (N,N-BIS(2-HYDROXY-ETHYL)-p-PHENYLENE-DIAMINE), 2,4,5,6-Tetraminopyrimidine (HC Red 16), 1-Hydroxy-3-methyl-4-aminobenzene (4-AMINO-m-CRESOL), 1-Hydroxy-2-amino-5-methylbenzene (6-AMINO-m-CRESOL), 1,3-Bis-(2,4-Diaminophenoxy)propane (1,3-BIS-(2,4-DIAMINO-PHENOXY)-PROPANE), 1-(2′-Hydroxyethyl)-2,5-diaminobenzene (HYDROXYETHYL-p-PHENYLENE DIAMINE SULPHATE), 1-Methoxy-2-amino-4-(2′-hydroxyethylamino)benzene, (2-AMINO-4-HYDROXYETHYLAMINOANISOLE) 1-Hydroxy-2-methyl-5-amino-6-chlorobenzene (5-AMINO-6-CHLORO-o-CRESOL), 1-Hydroxy-2-amino-6-methylbenzene (6-AMINO-o-CRESOL), 1-(2′-Hydroxyethyl)-amino-3,4-methylenedioxybenzene (HYDROXYETHYL-3,4-METHYLENEDIOXY-ANILINE HCl), 2,6-Dihydroxy-3,4-dimethylpyridine (2,6-DIHYDROXY-3,4-DIMETHYLPYRIDINE), 3,5-Diamino-2,6-dimethoxypyridine (2,6-DIMETHOXY-3,5-PYRIDINEDIAMINE), 5,6-Dihydroxyindole (DIHYDROXY-INDOLE), 4-Amino-2-aminomethylphenol (2-AMINOETHYL-p-AMINO-PHENOL HCl), 2,4-Diamino-5-methylphenetol (2,4-DIAMINO-5-METHYL-PHENETOLE HCl), 2,4-Diamino-5-(2′-hydroxyethyloxy)toluene (2,4-DIAMINO-5-METHYLPHENOXYETHANOL HCl), 5-Amino-4-chloro-2-methylphenol (5-AMINO-4-CHLORO-o-CRESOL), 4-Amino-1-hydroxy-2-(2′-hydroxyethylaminomethyl)benzene HYDROXYETHYLAMINOMETHYL-p-AMINO PHENOL HCl), 4-Amino-1-hydroxy-2-methoxymethylbenzene (2-METHOXYMETHYL-p-AMINOPHENOL HCl), 1,3-Bis(N(2-Hydroxyethyl)N(4-amino-phenyl)amino)-2-propanol (HYDROXYPROPYL-BIS-(N-HYDROXY-ETHYL-p-PHENYLENEDIAMINE)HCL), 6-Hydroxyindole (6-HYDROXY-INDOLE), 2,3-Indolinedione (ISATIN), 3-Amino-2-methylamino-6-methoxypyridine (HC BLUE NO. 7), 1-Phenyl-3-methyl-5-pyrazolone-2,4-dihydro-5,2-phenyl-3H-pyrazole-3-one, 2-Amino-3-hydroxypyridine (2-AMINO-3-HYDROXYPYRIDINE), 5-Amino-salicylic acid, 1-Methyl-2,6-bis(2-hydroxy-ethylamino)benzene (2,6-HYDROXYETHYLAMINO-TOLUENE), 4-Hydroxy-2,5,6-triaminopyrimidine (2,5,6-TRIAMINO-4-PYRIMIDINOL SULPHATE), 2,2′-[1,2-Ethanediyl-bis-(oxy-2,1-ethanediyloxy)]-bis-benzene-1,4-diamine (PEG-3,2′,2′-DI-p-PHENYLENEDIAMINE), 5,6-Dihydroxyindoline (DIHYDROXYINDOLINE), N,N-Dimethyl-3-ureidoaniline (m-DIMETHYL-AMINO-PHENYLUREA), 2,4-Diamino-5-fluortoluenesulfatehydrate (4-FLUORO-6-METHYL-m-PHENYLENEDIAMINE SULPHATE) and 1-Acetoxy-2-methylnaphthalene (1-HYDROXYETHYL-4,5-DIAMINOPYRAZOLE SULPHATE). These can be used in the molecular form or in the form of peroxide-compatible salts.

The hair colouring compositions of the present invention may also include non oxidative hair dyes. i.e. direct dyes which may be used alone or in combination with the above described oxidative dyes. Suitable direct dyes include azo or anthraquinone dyes and nitro derivatives of the benzene series and or melanin precursors and mixtures thereof. Such direct dyes are particularly useful to deliver shade modification or highlights. Particularly preferred are Basic Red 51, Basic Orange 31, Basic Yellow 87 and mixtures thereof.

C. Alkalizing Agent

According to the present invention the composition may also comprise at least one source of alkalizing agent. Any agent known in the art may be used such as alkanolamides for example monoethanolamine, diethanolamine, triethanolamine, monopropanolamine, dipropanolamine, tripropanolamine, 2-amino-2-methyl-1,3-propanediol, 2-amino-2-methyl-1-propanol, and 2-amino-2-hydroxymethyl-1,3-propanediol and guanidium salts, ammonium chloride, ammonium sulphate, ammonium nitrate, ammonium phosphate, ammonium acetate, ammonium carbonate, ammonium hydrogen carbonate, ammonium carbamate, ammonium hydroxide, percarbonate salts, ammonia, ammonium carbonate, ammonium carbamate, ammonia, sodium silicate, sodium metasilicate, sodium disilicate, ammonium persulfates, sodium persulfate, potassium persulfate and mixtures thereof.

The compositions of the present invention may comprise from about 0.1% to about 40% by weight, preferably from about 1.0% to about 35%, most preferably from about 2% to about 30% of an alkalizing agent, preferably ammonium ions.

D. Oxidizing Agent

The compositions may comprise an oxidizing agent, present in an amount sufficient to bleach melanin pigment in hair and/or cause formation of dye chromophores from oxidative dye precursors (including developers and/or couplers when present). Typically, such an amount ranges from about 1% to about 20%, preferably from about 3% to about 15%, more preferably from about 6% to about 12%, by weight, of the developer composition. Inorganic peroxygen materials capable of yielding hydrogen peroxide in an aqueous medium are preferred and include but are not limited to: hydrogen peroxide; inorganic alkali metal peroxides (e.g. sodium periodate and sodium peroxide); organic peroxides (e.g. urea peroxide, melamine peroxide); inorganic perhydrate salt bleaching compounds (e.g. alkali metal salts of perborates, percarbonates, perphosphates, persilicates, and ammonium persulfates, sodium persulfate, potassium persulfate and mixtures thereof), which may be incorporated as monohydrates, tetrahydrates, etc.; alkali metal bromates; enzymes; and mixtures thereof. Preferred are hydrogen peroxide and persulphates. The persulfate powders may be mixed with another liquid hair treatment composition as described herein after or alternatively may be immobilized physically via applying the powder particles to the interior and/or exterior surface of the absorbent substrate in such a means that they are physically contained and do not exit the substrate easily under gravity. This can be achieved via a hollow pocket or reservoir within the absorbent substrate that contains the persulfate salt blend. The persulfate powder can be loaded to this reservoir by a number of mechanical means including gravity feed, air pressure feed and screw feed. Alternatively, the powder particles can be adhered to the surfaces via an anhydrous solvent or via a thin layer of a material such as polyvinyl alcohol that does not solubilize the salts, the mixture being left to dry on the surface to enable future shelf stability.

E. pH Modifiers and Buffering Agents

The compositions may further comprise a pH modifier and/or buffering agent in an amount that is sufficiently effective to adjust the pH of the composition to fall within a range from about 3 to about 13, preferably from about 8 to about 12, more preferably from about 8 to about 11. The preferred pH range for the carbonate ion source as described herein below is from 8.5 to 9.5, preferably from 8 to 9. Suitable pH modifiers and/or buffering agents for use herein include, but are not limited to: ammonia, alkanolamides such as monoethanolamine, diethanolamine, triethanolamine, monopropanolamine, dipropanolamine, tripropanolamine, tripropanolamine, 2-amino-2-methyl-1-propanol, and 2-amino-2-hydroxymethyl-1,3,-propandiol and guanidium salts, alkali metal and ammonium hydroxides and carbonates, preferably sodium hydroxide and ammonium carbonate, and acidulents such as inorganic and inorganic acids, e.g., phosphoric acid, acetic acid, ascorbic acid, citric acid or tartaric acid, hydrochloric acid, and mixtures thereof.

F. Carbonate Ion Source

The compositions of the present invention may further comprise in a preferred embodiment at least one source of peroxymonocarbonate ions, preferably formed insitu from a source of hydrogen peroxide and a carbonate ion source. According to the present invention the compositions thus also may comprise at least a source of carbonate ions or carbamate ions or hydrocarbonate ions or any mixture thereof. Any source of these ions may be utilized. Suitable sources for use herein include sodium, potassium, guanidine, arginine, lithium, calcium, magnesium, barium, ammonium salts of carbonate, carbamate and hydrocarbonate ions and mixtures thereof such as sodium carbonate, sodium hydrogen carbonate, potassium carbonate, potassium hydrogen carbonate, guanidine carbonate, guanidine hydrogen carbonate, lithium carbonate, calcium carbonate, magnesium carbonate, barium carbonate, ammonium carbonate, ammonium hydrogen carbonate and mixtures thereof. Percarbonate salts may also be utilized to provide both the source of carbonate ions and oxidizing agent. Preferred sources of carbonate ions, carbamate and hydrocarbonate ions are sodium hydrogen carbonate, potassium hydrogen carbonate, ammonium carbamate and mixtures thereof. The compositions of the present invention may comprise from about 0.1% to about 15%, preferably from about 0.1% to about 10% by weight, more preferably from about 1% to about 8% by weight of the carbonate ion.

G. Radical Scavenger System

The compositions may comprise a radical scavenger, in a sufficient amount to reduce damage to the hair during the coloring process. Typically, such an amount will range from 0.1% to 10%, preferably from 1% to 7%, by weight of the composition. The radical scavenger is preferably selected such that it is not an identical species as the alkalizing agent. The radical scavenger is a species that can react with a carbonate radical to convert the carbonate radical by a series of fast reactions to a less reactive species. Preferred radical scavengers may be selected from the classes of alkanolamines, amino sugars, amino acids and mixtures thereof, and may include, but are not limited to: monoethanolamine, 3-amino-1-propanol, 4-amino-1-butanol,5-amino-1-pentanol, 1-amino-2-propanol, 1-amino-2-butanol, 1-amino-2-pentanol, 1-amino-3-pentanol, 1-amino-4-pentanol, 3-amino-2-methylpropan-1-ol, 1-amino-2-methylpropan-2-ol, 3-aminopropane-1,2-diol, glucosamine, N-acetylglucosamine, glycine, arginine, lysine, proline, glutamine, histidine, serine, tryptophan and potassium, sodium and ammonium salts of the above and mixtures thereof. Other preferred radical scavenger compounds include benzylamine, glutamic acid, imidazole, di-tert-butylhydroxytoluene, hydroquinone, catechol and mixtures thereof.

H. Chelants

The compositions may comprise chelants in an amount sufficient to reduce the amount of metals available to interact with formulation components, particularly oxidizing agents, more particularly peroxides. Typically such an amount will range from at least about 0.25%, preferably at least about 0.5%, by weight, of the composition. Suitable chelants for use herein include but are not limited to: diamine-N,N′-dipolyacid, monoamine monoamide-N,N′-dipolyacid, and N,N′-bis(2-hydroxybenzyl)ethylenediamine-N,N′-diacetic acid chelants (preferably EDDS (ethylenediaminedisuccinic acid)), carboxylic acids (preferably aminocarboxylic acids), phosphonic acids (preferably aminophosphonic acids) and polyphosphoric acids (in particular straight polyphosphoric acids), their salts and derivatives.

I. Humectants

The compositions may comprise humectants. Typically the amount of humectants in the hair treatment composition will range from at least about 1 to about 50%, preferably from about 5 to about 40%, and more preferably from about 10 to about 30%, by weight, of the composition. Suitable humectants for use herein include but are not limited to: polyhydric alcohols such as glycerin, sorbitol, xylitol, butylene glycol, polyethylene glycol, and propylene glycol, and mixtures thereof. Preferably the humectant is glycerin.

J. Thickening Agents

The hair treatment composition may further comprise a thickening agent. The thickening agent is present at a level of from about 0.01% to about 20%, preferably from about 0.1% to about 10%, more preferably from about 0.3% to about 5%, and even more preferably from about 0.5% to about 3%, by weight of the composition.

Gel network thickener system may be used as thickening agent for the purpose of the present invention. The gel network thickener system comprises at least one low HLB surfactant or amphophile having high melting point and at least one additional second surfactant.

The low HLB surfactant or amphophile has an HLB of 6 or less and a melting point of at least 30° C. Representative examples include following compounds (in the examples below “solid” refers to material state at temperature below 30° C.): solid fatty alcohols, solid oxyethylenated fatty alcohols, solid glycol esters, solid oxyethylenated alkyl phenols, solid sorbitan esters, solid sugar esters, solid methyl glucoside esters, solid polyglycerine esters, solid alkyl glyceryl ethers, solid propylene glycol fatty acid esters, cholesterol and ceramides. Preferably the low HLB surfactants are selected from linear or branched fatty alcohols comprising from about 14 to 30 carbon atoms, oxyethylenated fatty alcohols comprising from about 16 to 30 carbon atoms and at most about 2 units of ethylene oxide and glycerol mono esters of fatty acids comprising from about 16 to 30 carbon atoms. Most preferably the low HLB surfactants include cetyl, stearyl, cetostearyl or behenyl alcohols, steareth-2 and glycerol monostearate.

The second surfactant of the gel network thickener system may be anionic, non-ionic or cationic.

Anionic surfactants are selected from surfactants according to the formula RnXmYM, wherein R is a alkyl, alkenyl or alkylaryl group having from 8 to 30 carbon atoms, X is a polar group comprising at least one carbon atom and at least one oxygen or nitrogen atom, Y is an anionic group selected from carboxylates, sulphates, sulphonates or phosphates, n and m are independently 1 or 2 and M is hydrogen or a salt forming cation and mixtures thereof. Representative examples of anionic surfactants include salts (such as alkaline salts, for example, sodium salts, ammonium salts, amine salts, amino alcohol salts and magnesium salts) of the following compounds: alkyl ether carboxylates, alkyl ether sulphates, alkyl glyceryl sulphonates, alkylamido ether sulphates, alkylarylpolyether sulphates, alkyl monoglyceride sulphates, alkyl ether sulphonates, alkylamide sulphonates; alkyl succinates, alkyl sulphosuccinates, alkyl ether sulphosuccinates, alkylamide sulphosuccinates; alkyl sulphosuccinamates; alkyl sulphoacetates; alkyl ether phosphates; acyl sarcosinates, N-acyl methylaminopropionate; acyl isethionates, N-acyltaurates; acyl lactylates; carboxyalkyl ether of alkyl polyglucosides; alkyl lecithin derivatives. The alkyl or acyl radical of all of these various compounds, for example, comprises from about 8 to 30 carbon atoms, and the aryl radical, for example, is chosen from phenyl and benzyl groups.

Nonionic surfactants suitable for use in the gel network thickener system include non-ionic surfactants having an HLB of 7 or more and comprising one or more polyethyleneoxide chains wherein each polyethyleneoxide chain contains on average at least about 50 ethylene oxide units. Also suitable for use as nonionic surfactants are non-ionic surfactants having an HLB of 7 or more which are free of polyethyleneoxide chains. Representative examples of non-ionic surfactants free of polyethyleneoxide chains include polyglycerolated fatty acids, polyglycerolated fatty amides, polyglycerolated alkyl phenols, polyglycerolated α-diols, polyglycerolated alcohols, alkyl polyglucosides and sugar esters. Preferably, the non-ionic surfactants free of polyethyleneoxide chains are selected from alkyl polyglucosides, sugar esters, polyglyceryl fatty acid esters, alkyl polyglyceryl ethers and mixtures thereof. Representative examples of non-ionic surfactants comprising one or more polyethyleneoxide chain wherein each polyethyleneoxide chain contains on average at least about 50 ethylene oxide units include the following compounds: polyoxyethylene alkyl ethers, polyethyleneglycol fatty acid esters, polyoxyethylene castor oil, polyoxyethylene hydrogenated castor oil, polyoxyethylene fatty amides and their momoethanolamine and diethanolamine derivatives and polyethoxylated fatty amines, with a number of ethylene oxide groups of at least about 50 and mixtures thereof.

The cationic surfactants suitable for use in the gel network thickener system are selected from quaternary ammonium salts or amido-amines having at least one fatty chain comprising from 8 to 30 carbon atoms and mixture thereof. The quaternary ammonium salts have general formula N+ (R1R2R3R4) X−, wherein, R1 is selected from linear and branched radicals comprising about 12 to 30 carbon atoms, R2 is selected from linear and branched radicals comprising about 12 to 30 carbon atoms or the same group as radicals R3 to R4, the radicals R3 to R4, which may be identical or different, are selected from linear and branched aliphatic radicals comprising from about 1 to 4 carbon atoms, and aromatic radicals such as aryl and alkylaryl, the aliphatic radicals may comprise at least one hetero atom such as oxygen, nitrogen, sulphur and halogens, the aliphatic radicals are chosen, for example, from alkyl, alkoxy and alkylamide radicals, and wherein X− is an anion selected from halides such as chloride, bromide and iodide) (C2-C6)alkyl sulphates, such as methyl sulphate, phosphates, alkyl and alkylaryl sulphonates, and anions derived from organic acids, such as acetate and lactate. The cationic surfactant is preferably selected from, for example, a behentrimonium chloride, behenamidopropyltrimonium methosulfate, stearamidopropyltrimonium chloride, arachidtrimonium chloride and mixtures thereof. The amido-amine have general formula R′1-CONH(CH2)nNR′2R′3: wherein, R′1 is selected from linear and branched radicals comprising about 12 to 30 carbon atoms, the radicals R′2 and R′3, which may be identical or different, are selected from hydrogen, linear and branched aliphatic radicals comprising from about 1 to 4 carbon atoms, and aromatic radicals such as aryl and alkylaryl, the aliphatic radicals may comprise at least one hetero atom such as oxygen, nitrogen, sulphur and halogens, the aliphatic radicals are chosen, for example, from alkyl, alkoxy and alkylamide radicals, and wherein n is integer from 1 to 4. The amido-amine is preferably selected from, for example, behenamidopropyldimethylamine, behenamidopropyldiethylamine, behenamidoethyldiethyl-amine, behenamidoethyldimethylamine, arachidamidopropyldimethylamine, arachidamido-propyldiethylamine, arachidamidoethyldiethylamine, arachidamidoethyldimethylamine, and mixtures thereof.

More than one surfactant of the above specified types or any combination of the surfactants can be used and the weight ratio of the low HLB surfactants to the second specified surfactants is from about 100:1 to about 1:10, preferably from 20:1 to 1:2, and more preferably from 10:1 to 1:1.

Amide surfactants are also suitable thickening agents, preferably when mixed with a source of carbonate ions. The amide surfactants may be selected from polyoxyethylene amides or polyhydroxy amides. Polyoxyethylene amides are selected from compounds according to the formula R—(OCH2CH2)x-(OCH2)y-C(O)NH(CH2CH2O)z-H, wherein x is independently selected from 0 to 100, y is 0 or 1, z is independently selected from 1 to 100, and R is independently selected from alkyl, alkenyl or alkylaryl groups having from 8 to 30 carbon atoms or is a polyhydroxy amide according to the formula:

RC(O)N(R′)CH2-(CHOH)n-H, wherein n is independently selected from 4 to 6, R′ is hydrogen or methyl group, R is independently selected from alkyl, alkenyl or alkylaryl groups having from 8 to 30 carbon atoms. Preferred materials are available from Hatco Corporation. A particularly preferred material is laurylmethyl glucamide (HATCO HXL 7658).

Worm-like micelle phase thickening systems are also suitable thickening agents for the purpose of the present invention. The worm-like micelle thickening system of the present invention is defined as a thickening system comprising at least one ionic surfactant and an electrolyte source of counter-ions for said ionic surfactant. Suitable ionic surfactants for use herein may be selected from anionic surfactants, cationic surfactants and or mixtures thereof. The anionic surfactant described above for the gel network thickener system can be, alone or as mixtures, for use herein. Among the anionic surfactants, which can also be used, mention may also be made of fatty acid salts such as the salts of lauric, myristic, oleic, ricinoleic, palmitic and stearic acids, coconut oil acid or hydrogenated coconut oil acid; acyl lactylates in which the acyl radical comprises from 8 to 20 carbon atoms. Weakly anionic surfactants can also be used, such as alkyl-D-galactosiduronic acids and their salts, as well as polyoxyalkylenated (C6-C24) alkyl ether carboxylic acids, polyoxyalkylenated (C6-C24) alkylaryl ether carboxylic acids, polyoxyalkylenated (C6-C24) alkylamido ether carboxylic acids and their salts, for example, those comprising from 2 to 50 ethylene oxide groups, and mixtures thereof. Anionic derivatives of polysaccharides, for example carboxyalkyl ether of alkyl polyglucosides, can be also used. Preferably, the anionic surfactants are selected from those having an alkyl or acyl radical comprising from 8 to 16 carbon atoms and a Kraft point of less than 25° C. Preferably, the anionic surfactants are selected from alkyl sulphates, alkyl phosphates, alkyl ether phosphates, alkyl ether sulphates, alkyl glyceryl sulphonates, N-acyl sarcosinates, N-acyl taurates, acyl lactylates and carboxyalkyl ether of alkyl polyglucosides and fatty acid salts and alkyl ether carboxylates and mixtures thereof. Yet more preferably, the anionic surfactants are selected from N-acyl sarcosinates, alkyl sulphates, alkyl phosphates, alkyl ether sulphates, alkyl ether phosphates with average 1 to 20, preferably 1-10 and most preferably 1-3 ethylene oxide units. The anionic surfactants may also be preferably selected from those having an alkyl or acyl radical comprising 16 carbon atoms with on average 1.6 methyl branches per molecule or at least one unsaturated carbon bond.

The cationic surfactants suitable for use in the worm-like micelle thickening system of the present invention may be selected from quaternary ammonium salts or amido-amines as described above for the gel networking thickener system.

The worm-like micelle phase thickening system comprises at least one electrolyte source of counter ions for the ionic surfactant. The term electrolyte as used herein refers to ionic salts or compounds that ionize in solution resulting in the presence of positive and negative ions. According to the present invention the term counter-ion as used herein refers to ions which are of opposite charge to that on the ionic surfactant used to form said system. For example, in an anionic surfactant based worm-like micelle thickening system, the counter ions are the cations. Suitable electrolytes for use in the present invention result in the thickening of the ionic surfactant at a first concentration and thinning of the surfactant at a second higher concentration. Suitable electrolytes for use herein include alkaliser sources such as ammonium ion sources, carbonate ion sources, radical scavengers sources and mixtures thereof. Suitable sources of alkalizer include sources of ammonium ions, for example ammonium chloride, ammonium sulphate, ammonium nitrate, ammonium phosphate, ammonium acetate and mixtures thereof. Suitable electrolytes for use herein include but are not limited to sodium, potassium, lithium, calcium, magnesium, barium, ammonium salts of carbonate, carbamate, percarbonate, hydrogencarbonate ions, sulphate, phosphate, salicylate, chloride, bromide, iodide, fluoride and mixtures thereof such as ammonium carbonate, ammonium hydrogen carbonate, potassium carbonate, sodium carbonate, sodium hydrogen carbonate, potassium hydrogen carbonate, lithium carbonate, calcium carbonate, magnesium carbonate, barium carbonate, sodium chloride, magnesium chloride, potassium chloride, sodium sulphate, potassium sulphate, magnesium sulphate, ammonium sulphate, sodium salicylate, potassium salicylate and mixtures thereof. Another preferred type of electrolyte is a radical scavenger source. Example of such radical scavengers include for example potassium, sodium and ammonium salts of glycine, sarcosine, lysine, serine, glutamic acid and mixtures thereof. Particularly preferred electrolyte source are selected from ammonium carbonate, ammonium hydrogen carbonate, ammonium carbamate, sodium glycinate or mixtures thereof. Sources of electrolyte may alternatively be provided from other components in the hair colourant and or bleach compositions such as surfactants, dyes, preservatives, antioxidants and residual salt present from any components in the composition.

Suitable thickening agents for use herein also include synthetic polymers such as cellulose derivatives (e.g. methylcellulose, carboxymethylcellulose, hydroxyethylcellulose, hydroxypropylcellulose, hydroxy-propylmethylcellulose, etc.), carbomer polymers (e.g. crosslinked polyacrylic acid copolymer or homopolymer and copolymers of acrylic acid cross linked with a polyalkenyl polyether), natural and synthetic gums, karaya gum, guar gum, gelatin, algin, sodium alginate, tragacanth, chitosan, polyethylene oxide, acrylamide polymers, polyacrylic acid, polyvinyl alcohol, polyamines, polyquarternary compounds, ethylene oxide polymers, polyvinylpyrrolidone, cationic polyacrylamide polymers, and mixtures thereof.

In one embodiment the thickening agent is selected from carbomers, e.g. the class of homopolymers of acrylic acid crosslinked with an alkyl ether of pentaerythritol or an alkyl ether of sucrose. Carbomers are commercially available from B.F. Goodrich as the Carbopol® series. In one embodiment the carbopols are Carbopol 934, 940, 941, 956, and mixtures thereof. Homopolymers of polyacrylic acid are described, for example, in U.S. Pat. No. 2,798,053. Other examples of homopolymers which are useful include Ultrez 10, ETD 2050, and 974P polymers, which are available from B.F. Goodrich Company. Such polymers are homopolymers of unsaturated, polymerizable carboxylic monomers such as acrylic acid, methacrylic acid, maleic acid, itaconic acid, maleic anhydride, and the like.

In another embodiment the thickening agent can be an associative thickener or stabilizer, such as a hydrophobically modified alkali soluble acrylic emulsion or a hydrophobically modified nonionic polyol polymer, i.e., a hydrophobically modified urethane polymer, hydrophobically modified ethoxylated urethane polymer or combinations thereof. Associative thickening agents may increase the retention or adhesion of compositions herein and/or integral carriers on the tooth surfaces, may slow the erosion of the compositions once applied on the tooth surfaces, and may improve the release of the compositions from the optional release liner disclosed herein.

Hydrophobically modified polyacrylic acid polymers are described, for example, in U.S. Pat. Nos. 3,915,921, 4,421,902, 4,509,949, 4,923,940, 4,996,274, 5,004,598, and 5,349,030. These polymers have a large water-loving hydrophilic portion (the polyacrylic acid portion) and a smaller oil-loving hydrophobic portion (which can be derived from a long carbon chain acrylate ester). Representative higher alkyl acrylic esters are decycl acrylate, lauryl acrylate, stearyl acrylate, behenyl acrylate and melissyl acrylate, and the corresponding methacrylates. It should be understood that more than one carboxylic monomer and more than one acrylate ester or vinyl ester or ether or styrenic can be used in the monomer charge. The polymers can be dispersed in water and neutralized with base to thicken the aqueous composition, form a gel, or emulsify or suspend a deliverable. Useful polymers are sold as Carbopol® 1342 and 1382, and Carbopol® ETD 2020, and Pemulen® TR-1, TR-2, 1621, and 1622, all available from BF Goodrich. The carboxyl containing polymers are prepared from monomers containing at least one activated vinyl group and a carboxyl group, and would include copolymers of polymerizable carboxylic monomers with acrylate esters, acrylamides, alkylated acrylamides, olefins, vinyl esters, vinyl ethers, or styrenics. The carboxyl containing polymers have molecular weights greater than about 500 to as high as several billion, or more, usually greater than about 10,000 to 900,000 or more.

Also useful are interpolymers of hydrophobically modified monomers and steric stabilizing polymeric surface active agents having at least one hydrophilic moiety and at least one hydrophobic moiety or a linear block or random comb configuration or mixtures thereof. Examples of steric stabilizers which can be used are Hypermerl, which is a poly(12-hydroxystearic acid) polymer, available from Imperial Chemical Industries Inc. and Pecosil®, which is a methyl-3-polyethoxypropyl siloxane-phosphate polymer, available from Phoenix Chemical, Somerville, N.J. These are taught by U.S. Pat. Nos. 4,203,877 and 5,349,030.

Other associative thickening agents include Rohm and Haas (such as Acrysol® ICS-1 and Aculyn® 22 and 28 thickeners, which are hydrophobically modified alkali-soluble acrylic polymer emulsions and Aculyn® 44 and 46 thickener, which is a hydrophobically modified nonionic polyol). In one embodiment the associative thickener is Carbopol® and/or Pemulen® polymers. The choice of the specific polymer to be employed will depend upon the desired rheology of the composition, and the identity of other compositional ingredients. Other associative thickeners are discussed in greater detail in U.S. Pat. No. 5,997,764, BF Goodrich. In one embodiment mixtures of hydrophobically modified carbomers with carbomers can be used.

Preferred thickening agents are chosen from polymers (including gelling agents), gel phases referred to as creams or emulsions and combinations thereof.

Suitable polymers may be selected from carboxymethyl cellulose, carboxypropyl cellulose, carboxypolymethylene (Carbomers, Carbopols e.g. Carbopol ETD 2020, all RTM), carboxyvinyl Polymers, poloxamers, polyethylene glycol, natural gums (including but not limited to carrageenan, tragacanth, karaya, arabic, guar and xanthan), natural and synthetic smectite clays (including but not limited to hectorites, bentonites and montmorillonites), scleroglucan, methyl cellulose, ethyl cellulose (commercially available as Aquacote (RTM)), hydroxyethyl cellulose (Natrosol, hydroxypropylmethyl cellulose, microcrystalline cellulose, hydroxybutylmethyl cellulose, hydroxypropyl cellulose (Klucel (RTM)), hydroxyethyl ethyl cellulose, cetyl hydroxyethyl cellulose (Natrosol (RTM) Plus 330), N-vinylpyrollidone (Povidone (RTM)), Acrylates/Ceteth-20 Itaconate Copolymer (Structure (RTM) 3001), hydroxypropyl starch phosphate (Structure (RTM) ZEA), polyethoxylated urethanes or polycarbamyl polyglycol ester (e.g. PEG-150/Decyl/SMDI copolymer=Aculyn (RTM) 44, PEG-150/Stearyl/SMDI copolymer=Aculyn 46 (RTM)), trihydroxystearin (Thixcin (RTM)) acrylates copolymer (e.g. Aculyn (RTM) 33) or hydrophobically modified acrylate copolymers (e.g. Acrylates/Steareth-20 Methacrylate Copolymer=Aculyn (RTM) 22).

A representative but not exhaustive list of polymers and thickening agents can be found in “The Encyclopaedia of Polymers and Thickeners for Cosmetics” compiled and edited by Robert Y. Lochhead, PhD and William R. Fron, Department of Polymer Science, University of Southern Mississippi.

Suitable gel phase referred to as creams or emulsions may be selected from cetyl alcohol, stearyl alcohol, fatty acids and mixtures thereof.

### 4. OPTIONAL APPLICATION TOOL

An application tool (20) can optionally assist the delivery of the hair treatment composition (15) when used in conjunction with the hair treatment application system (10) according to the invention.

The application tool (20) provides ease of handling of the absorbent substrate (30) by the user. In addition, the application tool (20) minimizes contact of the user with the hair treatment composition (15) and also improves the application process of the hair treatment composition (15) to the hair. Preferably, the application tool (20) may allow exact selection of the hair strand to be treated and may be independent of orientation. The application tool (20) may provide means of separating one from more hair treatment compositions (15) e.g. oxidising agent and alkalising agent prior to activation and mixing to form a highlighting composition. The application tool (20) may help provide partial or full containment of one or more hair treatment compositions (15) within the hair treatment application system (10). Moreover, the application tool (20) may aid the delivery of the hair treatment composition (15) onto or in close proximity to the absorbent substrate (30). The application tool (20) may further provide a way to store the hair treatment composition (15) over the required product shelf-life.

Suitable application tools (20) include, but are not limited to, tools composed by a single piece or by a plurality of same or different parts. Examples include, but are not limited to hair hooking tool and sleeve, tubular dual-compartment tool with a pull-through device, plates attached to handles, tongs, applicators to be fixed to the user's finger, flexible pliable foils, filmic or cellulose based substrates or carrier materials, clips, clamps, shells, pincers, tweezers, scissors, single or multiple moulded parts with flexible elastomers or live hinges, folding combs, permeable or not permeable materials, interconnected plates (22), preferably hinged plates (22), as shown in FIG. 5. The plates (22) may present curved depressions on their surfaces and may include permanently connected or removable compartments (40) for the hair treatment composition (15).

The optional application tool (20) may also be provided with one or more means suitable to attach, adapt or install the dispensing or loading devices to perform the loading of the hair treatment composition (15) into the absorbent substrates (30) or into the application tool (20). Examples of said means are, but not limited to, nozzles, orifices, valves, one-way valves, sachets, single and dual phase pumps, single and dual phase pistons that volumetrically displace the liquid and syringes.

The application tool (20) may be manufactured from any known material capable of supporting the absorbent substrate (30) and the hair treatment composition (15). Suitable materials are a polymer resin such as a polyolefin e.g. polypropylene, polyethylene or polyethylene terephthalate. Other polymers could be used including polyvinylchloride, polyamide, acetyl, acrylonitrile butadiene styrene, acrylic, acrylonitrile styrene acrylate, ethylene vinyl alcohol, polycarbonate, polystyrene, silicone or thermo plastic elastomer or copolymers where appropriate or a flexible pliable substrate such as paper, board, metal based substrates and aluminium foil, filmic substrates or multiple laminations or combinations of multiple layers of said materials.

The application tool (20) or at least a portion thereof should preferably fit within the user's hand and it is preferably ergonomically designed to better adapt to the shape of the human hand for ease of use during application when used in either hand.

The application tool (20) comprises surfaces for attachment or association with the absorbent substrate (30). The application tool (20) has at least one surface to which one or more absorbent substrates (30) can be attached. Preferably, the surfaces of the application tool (20) extend beyond the surface of the absorbent substrate (30).

The application tool (20) is removable or permanently attached to the absorbent substrates (30). Any methods suitable to attach the absorbent substrate (30) to the application tool (20) may be employed herein providing that said method does not destroy or alter the performance of the absorbent substrate (30). The absorbent substrate (30) may be attached to the application tool (20) by using heat welding including pressure, ultrasonic forces, radio or high frequencies. The absorbent substrate (30) may also be attached to the application tool (20) through adhesive, including two-side tape, thermo-set, hot melt and cold seal, adhesion or extrusion lamination. Mechanical interlock or entanglement such as Velcro®, clamping, snap locks, sealing beads, locking pins and magnetism may also be used to adhere the absorbent substrate (30) to the application tool (20). The hair treatment composition (15) may be loaded before or after the adhesion of the absorbent substrate (30) to the application tool (20), but for those methods that could alter or inactivate the hair treatment composition (15), the absorbent substrate (30) is loaded after it has been attached to the application tool (20).

The optional application tool (20) may comprise one or more of the same or different hair treatment compositions (15) contained in one or more of its parts. Examples include, but are not limited to, compartments (40) separated by barrier material or liners removable via peeling, rupturing, puncturing, breaking, tearing, piercing, sliding, folding, compression, ball bearing. Said compartments (40) may be fixed, attachable, removable and may be disposed once the hair treatment compositions (15) have been released. Different hair treatment compositions (15) may come into contact through fluid inter-mixing mechanisms either prior to or during usage including, but not limited to, dispensing from two different compartments (40) through a torturous pathway via pressure, shearing static mixers, porous media, perforated solids, sintered materials, coated materials, laminated structures or any combination of those mechanisms.

The hair treatment compositions (15) may be dispensed simultaneously or sequentially. The hair treatment compositions (15) may be dispensed in close proximity to the absorbent substrate (30) or into a void space within the application tool (20). In one embodiment shown in FIG. 2, the application tool (20) is formed by a single elongated piece having a handle (21) on one side and a plate (22) on the other side to which the hair treatment application system (10) is adhered.

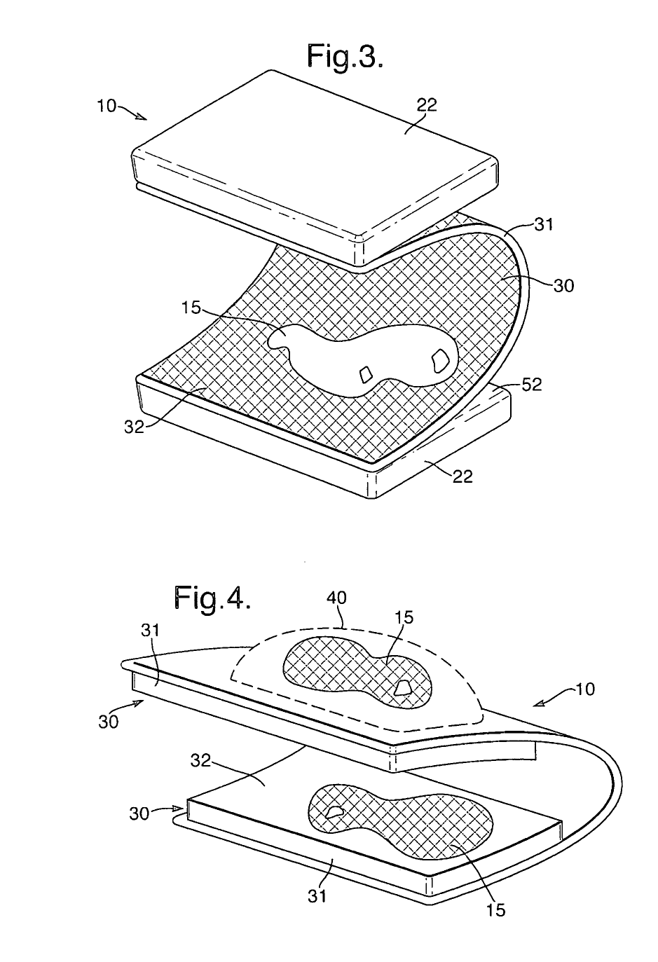

In another embodiment for use herein the application tool (20) is formed by a single piece obtained by moulding a flexible and pliable support as shown in FIG. 4. In one preferred embodiment the application tool (20) comprises two plates (22), preferably connected via any suitable means, such as the absorbent substrate itself (30) as shown in FIG. 3 or via a hinge (50) as shown in FIG. 5. The hinge (50) can be formed in a number of ways including: a “live” injection moulded hinge, a strap hinge or flexible strip (folding link that is connected between the two components), a kiss-cut or a crease.

The plates (22) may be flat or curved and each plate (22) has an external (51) and internal (52) surface. In the preferred embodiment, an absorbent substrate (30) is attached to the internal surface (52) of each plate (20), via its outer surface (31). The plates (22) may be identical in design for ease of manufacture and assembly, but in other embodiments the plate's design may be different. Both the first and second plates (22) may be manufactured in a number of ways including: injection moulding, bi-injection moulding, thermo or vacuum forming of a blister type shell, lamination onto a carrier plastic or board material in the horizontal plane.

The hair treatment composition (15) can be pre-impregnated in the absorbent substrates (30), pre-loaded either in the absorbent substrate (30) or in one or more compartments (40) of the application tool (20) or the hair treatment compositions (15) can be applied just prior to use in the entirety or just a portion of the absorbent substrates (30) or into the application tool's compartments (40). In either case, the hair treatment compositions (15) may be identical or different. In one embodiment of the present invention, the absorbent substrate (30) may be provided with a release liner to protect the absorbent substrate (30) itself and or the hair treatment composition (15) from contamination when not in use. The release liner may be resealed after use to the absorbent substrate (30), or to the application tool (20), if present. The release liner may be aluminium or plastic (low density polyethylene/aluminium laminate/polyethylene terephthalate) peel-able foils and may be made of a gas resistant material, especially for hair treatment composition (15) comprising hydrogen peroxide. When the absorbent substrate (30) are attached to an optional application tool (20) said absorbent substrates (30) can be protected by external contamination by closure or covering means applied or mounted on the application tool (20) itself such as screwable caps, which are removed during use but that may be easily reapplied to protect the hair treatment composition (15) and the absorbent substrates (30) after use. Such closure or covering means serve also to avoid contamination of the user's home with the hair treatment composition (15) comprised therein when the application tool (20) is laid down during use.

### 5. HAIR TREATMENT COMPOSITION LOADING AND MIXING

According to the present invention said one or more hair treatment compositions (15) may be loaded into at least one absorbent substrate (30). In embodiments where only one absorbent substrate (30) is present, one or more hair treatment compositions (15) may be applied either on the same or, preferably, on different sections of the absorbent substrate (30). In embodiments where more than one substrate is present, said absorbent substrates (30) may be the same or different and one or more hair treatment compositions (15) may be applied onto the same or different sections of each absorbent substrate (30).

When the optional application tool (20) is present the hair treatment composition (15) may be loaded into the application tool (20), preferably into an application tool's separate compartment (40) or within a void space of the application tool (20).

The hair treatment composition (15) can be loaded onto the absorbent substrate (30) or into the application tool (20) by any means. In one embodiment the hair treatment composition (15) is loaded into the absorbent substrate (30) directly by applying the hair treatment composition (15) for example with a spatula or a syringe, by a squeezable tube, by a dispensing bottle, by a single or dual phase pump, by a single or dual phase piston causing volumetric displacement, by a sachet or by any other suitable dispenser as shown in FIG. 6. In embodiments where the an application tool (20) is present, the loading may be performed by attaching or adapting the application tool (20) itself or parts of the application tool (20) to external loading devices such as squeezable tube, a bottle or a syringe or by any other suitable dispenser as described above. When the hair treatment compositions (15) require mixing and activation prior application to the hair, the single component of the hair treatment composition (15) may be mixed by shaking or stirring before loading or can be mixed during the loading procedure by employing specialized two or multi-chambered containers coupled with a static mixer. The mixing may also be performed by interposing an additional mean capable of mixing two or more hair treatment compositions (15) or capable of mixing powders with water or other solvents to make a hair treatment composition (15). Said interposed mean can also be provided with features to inject or load the mixed hair treatment compositions (15) onto the absorbent substrates (30) or into the application tool (20).

In another embodiment, upon the application of the hair treatment composition (15) into at least one absorbent substrate (30), the absorbent substrate (30) is subjected to mechanical pressure, preferably said pressure applied by the fingers of the user as shown in FIG. 1 (left), to allow the hair treatment composition (15) to evenly distribute within the whole absorbent substrate (30) by flowing through its pores.

One or more absorbent substrates (30) or application tools (20) can be loaded at the same time or one subsequently to the others. Multiple or subsequent loading may be accomplished by positioning, the absorbent substrates (30) or the application tools (20) in a tray or by connecting or attaching the absorbent substrates (30) or the application tools (20) to multi-chambered bottles, tubes or other applicators capable of dispensing either the single or the total amount of the needed hair treatment composition (15). When a tray is used, said tray comprises at least one compartment where the absorbent substrates (30) or the application tools (15) are positioned or adapted. The tray may further comprise one or more compartments where the hair treatments compositions (15) are loaded and/or stored and which are in communication with the absorbent substrates (30) or the compartments of the application tool (20) where the hair treatment composition (15) has to be contained.

The hair treatment compositions (15) may be loaded by pressure or by vacuum. When vacuum is used, the air within the absorbent substrate (30) is displaced by the hair treatment composition (15). In the case of pressure, the hair treatment composition (15) is moved into the absorbent substrate (30) or into a compartment of the application tool (20) by means of an air pump, hand-bellows or alike. The same or different hair treatment compositions (15) can be loaded in different sections of the absorbent substrates (30) or in different compartments (40) of the application tool (20). The hair treatment compositions (15) may be loaded in different time, such as one hair treatment composition (15) is loaded and only when that hair treatment composition (15) is absorbed evenly within the absorbent substrate (30), then a second and any further hair treatment compositions (15) are applied.

Any of the above described method of loading and applying the hair treatment composition (15) can be either performed manually or by mechanically operated machines, especially when the loading is executed during manufacture of the hair treatment application system (10). One or more methods may be employed either simultaneously or subsequently one to another.

In another embodiment, the hair treatment composition (15) may be loaded into a vacuum formed or blister tray which is sealed by a higher barrier laminate material.

The amount of hair treatment composition (15) applied on the absorbent substrates (30) will depend upon the size and capacity of the absorbent substrate (30), type of hair treatment composition (15) and the desired end results.

### 6. METHOD OF USE

The present invention also relates to a method to treat the hair by contacting the hair with the hair treatment application system (10). By contacting the hair with the hair treatment application system (10) the hair treatment composition (15) is delivered to the hair. The user may hold the hair treatment application system (10) on the absorbent substrate's outer surface (31) as shown in FIG. 6. The inner surface (32) of the absorbent substrate (30) is the surface that is brought into contact to the hair. The user contacts the hair with the absorbent substrate's (30) inner surface (32) as shown in FIG. 1 (right). To perform this action the user may simply contact the absorbent substrate (30) to the hair, preferably at the root-line and coat the hair treatment composition (15) as shown in FIG. 2. Otherwise the absorbent substrate (30) may be folded along one of its dimensions as shown in FIG. 1 so that the inner surface (32) can be wrapped around a hair strand (11) while the user holds in only one hand the absorbent substrate's (30) outer surfaces (31), preferably the user holds the absorbent substrate (30) between the thumb and the index finger. The selected hair strand (11) is held with the other hand while the absorbent substrate (30) is positioned onto the hair strand (30), preferably at the root line. The hair treatment composition (15) is then applied by swiping the hair treatment applicator system (10) along the entire length of the selected hair strand (30) as shown in FIG. 1.

In the embodiments of the hair treatment application system (10) as shown in FIGS. 4 and 5, comprising two absorbent substrates (30), the user selects the hair strand (11) and places it between the two absorbent substrates (30), preferably at the root line. The absorbent substrates (30) are clamped around the hair strand (11) and then moved along the length of the hair to the tips in order to apply the hair treatment composition (15). In one embodiment of the hair treatment application system (10) according to the invention, a first and a second absorbent substrates (30) are present. The first absorbent substrate (30) comprises a first hair treatment composition (15) and a second absorbent substrate (30) comprises a second hair treatment composition (15). The first and second hair treatment composition (15) may be the same or different. Preferably, when the first and second hair treatment compositions (15) are different, the first hair treatment composition (15) is capable of reacting with the second hair treatment composition (15) to form a third hair treatment composition (15). When the first and second compositions (30) are brought into contact, the hair treating composition (15) may be formed immediately or a further activation step may be required. For example the reaction may be heat-, water- or pressure-activated. In the case of water-activation, water may be applied to the hair prior to being placed between the absorbent substrates (30). In another embodiment the absorbent substrate (30) itself may be contacted with water prior to being applied on the strands of hair. Combination of the activation steps described above is also foreseeable. The mixing of said first and second hair treatment composition (15) is achieved by compressing and squeezing once, preferably more than once, said first and second absorbent substrates (30) before the application of the hair treatment composition (15) occurs.

In the most preferred embodiment two absorbent substrates (30) are attached to two hinged plates (22) and a hair strand (11) is then selected and located between the two absorbent substrates (30). Application of the hair treatment composition (15) loaded into the absorbent substrates (30) or mixing of the hair treatment compositions (15) loaded into the absorbent substrates (30) is obtained by applying a force on the external surface (51) of both plates (22) so that they swing by a certain degree with respect to their hinge (50) as shown in FIG. 5. This mixing, by pressing the two plates (22) together, compresses and squeezes the attached absorbent substrates (30) and forces the hair treatment compositions (15) contained therein to mix. This mixing method can be performed prior or during application to hair as many times as needed to evenly mix the hair treatment compositions before to proceed with the application onto the hair strand (11). In this most preferred embodiment, the absorbent substrate (30) that is attached to the first plate (22) is designated as an area to which a hydrogen peroxide based gel is loaded, whereas the absorbent substrate (30) that is attached on the second plate (22) is designated as an area to which an alkaliser gel is loaded. When the hydrogen peroxide gel and the alkalizer gel are mixed together they form a highlighting composition. It is irrelevant which plate (22) is designated as the first and second plate (22) as long as the hydrogen peroxide gel and the alkalizer gel are loaded in different absorbent substrates (30) or different sections thereof.

In one embodiment, a first hair treatment composition (15) can be incorporated within a single absorbent substrate (30) and is capable of reacting with a second hair treatment composition (15) that is applied to the hair separately to the application of the first hair treatment composition (15), preferably on a separate absorbent substrate (30). The first and the second hair treatment compositions (15) may be contacted together physically either immediately prior or during consumer application to the hair.

In another embodiment a first hair treatment composition (15) is applied to the hair from the absorbent substrate (30) as a pre- or post-treatment to a second hair treatment composition (15) and any further hair treatment composition (15) which can be applied via conventional liquids dispensed from bottles, as a cream, as a mousse, as a gel etc or with an additional hair treatment conventional device or an hair treatment application system (10) of the present invention.

As shown in FIGS. 1 and 2, the absorbent substrates (30) of the present invention are not intended to be applied to hair in a stationary manner but rather they are moved against the hair surface with the use of shear forces, i.e., swiping of individual hair strands, rubbing along root-line, rubbing into hair, wiping surface of hair, pulled through hair etc., thereby depositing the hair treatment composition (15) evenly along the entire length of the hair as required.

In embodiments where a liner is present, the user peels off the liner, loads the absorbent substrate (30) and applies the hair treatment composition (15) to the hair. During the application one or more liners can be resealed to protect the absorbent substrates (30) and the hair treatment compositions (15) or to avoid contamination of the user's home furniture with the hair treatment composition (15). In embodiments when the absorbent substrates (30) are pre-loaded by the manufactures and then sealed with liners, the user removes the liners before application of the hair treatment compositions (15). Once the liners have been removed, the user can either proceed to use or apply additional hair treatment compositions (15) by loading them onto the absorbent substrates (30) or into the optional application tool (20) as described above. One or more liners can be peeled off simultaneously or one individually after the other.

Finally, the application of the hair treatment composition (15) may occur on wet or dry hair and optionally, a rinsing or a shampooing step can be included between application of the first and any further compositions (15) to the hair.

### 7. KIT-OF-PARTS

The hair treatment application system (10) according to the present invention may be provided as a component of a kit-of-parts comprising single packaged containers as described herein below. The kit-of-parts according to the invention may comprise one or more individually packaged compositions comprising shampoo compositions, conditioning compositions, styling compositions, hair colourant compositions, hair bleaching, highlighting compositions or combination thereof. In one embodiment of the present invention, a first container may comprise an oxidative dye precursors and an alkalizing agent whereas a second container may comprise an oxidizing agent. In certain other embodiments of the kit-of-parts, a first container may comprise an alkalizing agent, preferably a source of ammonium ions and a second container may comprise an oxidizing agent, preferably hydrogen peroxide. Additional containers may be present in the kit-of-parts, such as individually packaged composition comprising additional components such as oxidising agents, conditioners, chelants, radical scavengers, solvents, direct dyes, shampoo, buffering agents, colouring agents thickeners, enzymes, anionic, non ionic, amphoteric and cationic surfactants, carriers, antioxidants, stabilizers, perfumes, masking fragrances, herb and plant extracts, pearlescent, opacifiers, hair swelling agents and/or polymers, humectants, moisturizers, viscosity enhancers, gelling agents, chelators, UV filters, antimicrobials, preservatives, proteins or mixtures thereof.

The kit-of-parts according to the present invention may further comprise additional components such as means to select the hair strand, means to load the hair treatment application system (10) according to the present invention, means to mix the hair treatment compositions (15), combs or brushes, gloves, caps with holes, tweezers, tongues, hooks or combination thereof.

In one embodiment the kit-of-parts according to the invention comprises the hair treatment application system (10) as described herein and gloves. In another embodiment the kit-of-parts further comprises an individually packaged composition comprising an oxidizing agent and an individually packaged composition comprising an alkalizing agent. Preferably said alkalizing agent comprises a persulfate salt.

In certain embodiments, the hair treatment application system (10) according to the present invention is provided with an optional application tool (20), The application tool (20) may be provided assembled but also unassembled in the kit-of-parts and instruction how to build the unassembled application tool (20) of the present invention may be further provided in the kit-of-parts described above. The kit-of parts comprising the hair treatment application system (10) according to the present invention may further comprise instructions for consumers indicating how to load and/or use the hair treatment application system (10), said instruction being recorded in any type of media such as the package of the kit-of-parts itself, paper material, compact disk or the hair treatment application system (10) itself or the optional application tool (20).

### 8. TEST METHODS

### Median Pore Radius

There is no single method to measure median pore radii across the range of absorbent substrate selected for the purpose of the present invention. The median pore radius of the absorbent substrate was measured firstly via TRI Autoporosimeter™. For values obtained above 1000 microns the measurement was repeated via optical imaging and the value obtained with the optical imagine was taken the final value.

The TRI Autoporosimeter™ is an automated, computer controlled instrument for measuring pore radii and their corresponding cumulative pore volumes (B. Miller and I. Tyomkin, Journal of Colloid and Interface Science, 162 (1994), 163-170). The median pore radius of a sample is the equivalent cylindrical radius where the cumulative volume of fluid absorbed/desorbed equals 50% of the saturation capacity of the test sample. The equivalent cylindrical radius can be afforded from the Laplace equation that relates the hydrostatic pressure (pressure at 50% saturation) to pore radius:

$$R = \frac{2 γcos θ}{Δ P}$$

where R is the effective radius, γ is the surface tension of wetting liquid, θ is the contact advancing or receding contact angle of liquid and ΔP is the pressure difference in hydrostatic head pressure across sample.

The median equivalent pore radius was typically determined from the desorption measurements, where θ is the receding contact angle. If the 50% value does not correspond to a pre-selected equivalent radius, then it can be determined graphically by interpolation.

The test sample, typically 50 mm in diameter, is placed in a measurement cell on a Millipore glass filter membrane (porosity of 1.2 microns) attached to a Monel support plate. The filter membrane and Monel metal support are prepared according to the manufacture's recommendations and attached via an epoxy based paint (Krylon High Gloss available from Swerin-Williams Corp.). Measurements are conducted with an applied pressure (confining weight) of 0.1 psi to ensure the test sample is in contact with the fluid test membrane.

The measurement cell is connected to a reservoir of the test liquid placed upon a balance. The TRI Autoporosimeter™ is used with n-hexadecane to wet the absorbent substrate. The weight (volume) of the liquid absorbed/desorbed by the absorbent substrate as a function of applied air pressure is recorded by the balance. As the air pressure increases and decreases, different size pore groups desorb and absorb liquid, respectively. The pore volume of each group is equal to this volume of liquid transferred. To ensure that all samples are tested under the same close to equilibrium conditions, the following parameters are recommended: (i) Equilibrium constant (liquid flow at each pressure step) is less than 90 mg/min, (ii) Balance weight data is collected every 15 seconds until desired equilibrium constant value is reached. The results are afforded as capillary pressure (cm), as a function of fluid weight on the balance (g).

Test samples with median pore radii greater than 1000 microns are determined by optical imaging. A test sample of 50×50 mm is placed in an Olympus Stereo-Microscope connected to a digital camera. The dark field illumination technique was used to enhance the contrast and improve the visual appearance of the 3D structure. The applied magnifications are chosen to a) obtain regions of interest (ROIs) that represent the structure of the samples and b) enable reliable measurements of the pore sizes. The pore area of all the focused pores are manually measured in the ROIs and subsequently analyzed with the Olympus analySIS FIVE software to afford the accumulative surface pore area as a function of equivalent circular pore radius. The median pore radius of a sample is the equivalent circular radius where the cumulative pore area equals 50% of the total surface pore area. The measurements include pores that are not completely parallel to the focal plane, but observed from a viewing angle (due to the 3D structure).

The median pore radius was reported to the nearest micron (μm). The median pore radii and the other technical characteristic of some of the absorbent substrates according to the present invention are presented in table 5 below. Table 5 also shows absorbent substrates that do not satisfy the specifications claimed herein.

### Absorption Capacity

The theoretical absorption capacity can be defined for a porous substrate as the mass of liquid absorbed per unit mass of dry solid substrate (see, for instance, Absorbent Technology, by Chatterjee and Gupta, Elsevier, 2002):

$$C = \frac{{ρ}_{l}}{ρ} \times \frac{φ}{1 - φ}$$

where ρ is the density of the material making up the substrate, ρ₁ is the density of the liquid (for the stated Absorption Capacities the density of pure water is used—1.00 g/cm³) and φ is 1 minus the quotient of ρ_{bulk} over ρ (ρ_{bulk} is the bulk density of the porous substrate). For multi-component fibers/blends and non-cylindrical fibers, a weighted average solid fiber density is used. The results were reported to the nearest gram of fluid per gram of substrate (g/g).

### Calliper

The calliper of the absorbent substrates is determined by the EDANA Recommended Test Method (ERT 30.5-99)—Thickness. An Abram Model 2000 micrometer with an accuracy of 0.01 mm and lowering speed of 3 mm/s and measuring pressure of 0.1 kPa was used (Technische Beratung Abram GmbH). The results were afforded to the nearest 0.01 mm. The same method was applied to measure the calliper of foam substrates.

### Basis Weight

The basis weight of the absorbent substrates is determined by the EDANA Recommended Test Method (ERT 40.3-90)—Mass per unit area. A substrate test area of 100×100 mm was accurately cut and weighed. The results reported as grams per square meter (g/m²). The same method was applied to measure the basis weight of foam substrates.

### Liquid Permeability Test Method

Liquid permeability is a measure of the resistance to flow in a porous medium and can be defined by the equation:

$$k = \frac{V μ L}{p}$$

where k is the liquid permeability, V is the fluid flow rate through the test sample, μ is the fluid viscosity, L is the calliper of the samples and P is the water column of known height along cross sectional area ρ×g×height (where ρ is the density of pure water—1.00 g/cm³ and g is the acceleration due to gravity).

The rate of fluid flow (V) under constant hydro head conditions is used to determine the through plane liquid permeability of a porous medium. The equipment consists of a column capable of maintaining a 10 to 250 mm constant hydro head by means of a fluid reservoir supply and variable flow tap. A fluid retention gate is located at the other end of the column. A circular test sample is cut to a diameter of 25.4 mm and secured within the column above the retention gate by an o-ring of known calliper. The measurement is conducted by opening the reservoir tap to fill the column to a height of 50 mm. The fluid retention gate is then opened and the reservoir tap adjusted as to maintain the constant fluid height. At least 100 cm³ of fluid must flow before the flow rate is determined by means of a computer linked balance. Deionised water was used in these measurements. The through plane liquid permeability can then be calculated and converted into Darcy units, where a Darcy unit=k×9.87×10⁻¹³.

A medium with a permeability of 1 Darcy permits a flow of 1 cm/s of a fluid with viscosity 1 cPs under a pressure gradient of 1 atm/cm.

TABLE 5
Technical characteristics of some absorbent substrates.

| | Median pore | Absorption capacity | Calliper | Basis weight | Liquid permeability |
|---|---|---|---|---|---|
| Absorbent substrate | radius [microns] | [g/g] | [mm] | [g/m²] | [Darcy Units] |
| Libeltex 01-766 DI-08 | 550 | 41 | 6.24 | 148 | 2457 |
| PGI FB-215 | 700 | 57 | 7.83 | 136 | 3924 |
| PGI FB-185 | 500 | 45 | 7.17 | 156 | 2325 |
| Recticel Bulpren Foam | 1400 | 40 | 6.02 | 143 | 5230 |
| S28280 | | | | | |
| Polyscorer Foam 410* | 2200 | 29 | 11.12 | 365 | 825 |
| PGI FB-213A | 700 | 41 | 4.94 | 118 | 2144 |
| BBA Fiberweb Tenotex | 70 | 7 | 0.46 | 59 | 25 |
| PL60L* | | | | | |
| Freundenberg | 300 | 16 | 0.99 | 60 | 595 |
| AL 1060 | | | | | |

### Mixing of Hair Treatment Compositions Method

When two or more hair treatment compositions are mixed prior to loading into the absorbent substrate the following procedure may be used. Said at least two hair treatment compositions, which may either be both liquid or one liquid and one powder, are placed into a vessel in a weight ratio between 1:10 and 10:1. This vessel may be of different forms, for example a bottle with a lid or an open compartment. In the case of the bottle, the two compositions are shaken for 2 minutes until the mixture is homogeneous. In the case of the open compartment the two compositions are mixed with a mixing implement for a period of time sufficient to produce a homogeneous mixed product. The mixed product produced via this method is the same as that obtained when the two compositions are loaded into separate absorbent materials and mixed as the absorbents are pressed together.

### Viscosity Measurement Test Method

The viscosity of the hair treatment composition to be loaded into the absorbent substrate was measured using a Brookfield viscometer with cone and plate attachment. For viscosities in the range of about 0 cPs to 12,000 cPs the Brookfield DV-II+ viscometer with S42 plate was used. A sample of 2 ml of the hair treatment composition or Carbopol 956 solution was equilibrated at 26.7° C. for one minute prior to measurement, whereupon the readings are taken at 1 rpm. For viscosity values of about 12,000 cPs, another measurement is taken as described herein below.

For viscosities in the range of 12,000 cPs to about 100,000 cPs the Brookfield DV-II+ viscosities with S52 plate was used. A sample of 0.5 ml of the hair treatment composition or Carbopol 956 solution was equilibrated at 26.7° C. for one minutes prior to measurement, whereupon the readings are taken at 1 rpm. Again as explained above for viscosity values of 100,000 cPs, another measurement is taken as described herein below.

For viscosities in the range of 100,000-150,000 cPs the Brookfield DV-II+ viscometer with S52 plate is used. A sample of 0.5 ml of the hair treatment composition or Carbopol 956 solution is equilibrated at approximately 26.7° C. for one minutes prior to measurement, whereupon the readings are taken at 0.5 rpm.

### Minimum Viscosity Limit Test

All absorbent substrates used in the following test methods had a calliper of from about 6 mm to about 10 mm. The required calliper was achieved for BBA Fiberweb Tenotex P101 and Freudenberg AL 1060 absorbent substrate by laminating multiply plies of the absorbent substrates by means of a hot melt adhesive positioned in circular droplets not greater than 2 mm of diameter on each surface.

The absorbent substrates were cut into circular pad of 3.57 cm of diameter (surface area 10.0 cm²), weighed and placed on the 4 cm diameter sinter glass disc of a 80 cm³ Pyrex Buchner funnel (available from Fisher; code FPJ-400-110D). The Carbopol 956 solution was carefully added onto the absorbent substrate to a depth of approximately 1 cm after which a hand bellow, attached with a single hole rubber bung fitting into the opening of the funnel, were inflated and used to force the fluid into the absorbent substrate for 1 minute. The absorbent substrate so loaded was removed carefully from the funnel with tweezers. Excess surface fluid was removed by placing two layers of laboratory tissue paper on a bench top and carefully passing the loaded absorbent (handled with tweezers) over the tissue for a distance of 30 cm, then turning the absorbent and repeating for the other side. The loaded absorbent substrate was weighed and centrally fixed by means of 3M double sided tape onto the flat side of the circular test plate of 4.97 cm diameter (made from rigid white acetal). The opposite plate was fitted with three brass pins appropriately selected such that the absorbent was compressed by 3 mm when the surfaces of the two plates are parallel and vertically compressed. The brass pin height was set to 3 mm for BBA Fiberweb Tenotex P101 and Libeltex 01-766 DI-8 whilst it was of 7 mm for Recticel Bulpren S28280. The plate containing the brass pins was secured above a weighing boat with its surface perpendicular to the lab bench and the empty fourth pin hole in the lowest position vertical to the top brass pin. The plate containing the absorbent substrate was then vertically compressed against the pin plate for 1 min. The weight of the weighing tray and of any dripped hair treatment composition or Carbopol 956 solutions was recorded and the percentage of the total fluid loaded that dripped out during the compression was calculated. The measurement was repeated three times for each experiment.

### Determination of the Minimum Viscosity Limit: Consumer Panellist Study

Libeltex 01-766 DI-8 absorbent substrate was cut into two circular pads of 3.57 cm diameter (surface area 10.0 cm²) and loaded with Carbopol 956 solutions having viscosities of 1,000 cPs 2,500 cPs or 5,000 cPs as described above. The loaded Libeltex 01-766 DI-8 absorbent substrate was centrally fixed to the opposing plates of the application tool by means of 3M double sided tape. The panellists were given the application tool so prepared and asked to swipe twice a 0.75 g bundle of human hair of 30.5 cm in length (Caucasion Light Brown—International Hair Imports and Products, Valhalla, N.Y.). Its performance was evaluated with the question “Considering the sample you have just used, how would you rate its mess?” Answers were rated on a 0 to 8 scale (0=not messy at all; 8=extremely messy).

### Minimum Median Pore Size Limit Test

The absorbent substrate samples (BBA Fiberweb Tenotex P101; Freudenberg AL 1060; Libeltex 01-766 DI-8; PGI FB-215 and Recticel Bulpren S28280), were cut into circular pads of 2.53 cm diameter (surface area 5.03 cm²), weighed and placed on the sintered glass filter disc in a 30 cm³ Duran Filter Assembly (Duran reference 24.720/24). A Viton ring was then placed upon the absorbent substrate such that the exposed absorbent had a diameter of 1.8 cm (2.54 cm²). The Duran Filter Assembly was attached and filled with the Carbopol 956 solutions to a depth of approximately 1 cm by slow addition down the side of the glass head avoiding absorbent disturbance. Additional Carbopol 956 solution was then poured into the glass head up to the graduated 30 cm³ mark. After 5 minutes the remaining fluid was separated and the absorbent substrates removed with tweezers. Any excess of surface fluid was removed by carefully passing the absorbent substrate over two layers of laboratory tissue paper a distance of 30 cm for each side. The loading (grams of Carbopol 956 solution per gram of absorbent substrate) was calculated based on the weight of the exposed absorbent substrate (surface area 2.54 cm²) as follows and reported to two decimal places:

$$Loading [ g \text{/} g ] = \frac{2 \times ( AS weight after loading ) - ( AS weight before loading )}{( AS weight before loading )}$$

wherein AS means absorbent substrate.

### Maximum Viscosity Limit

The Recticel Pottscorer 410 and Recticel Bulpren S28280 foams were cut into two circular pads of 3.57 cm diameter (surface area 10.0 cm²) and loaded with a 100,000 cPs model test fluid as described in the determination of the minimum viscosity limit method. The loaded pads were then centrally fixed, by means of 3M double sided tape, onto the flat sides of two test plates of 4.97 cm diameter. One of the plates was fitted with three 12 mm pins and secured on a clamp stand with the loaded Recticel Pottscorer 410 foam's surface perpendicular to the lab bench and the empty fourth pin hole at the lowest position. A 0.75 g bundle of hair (30.5 cm in length fanned to a width of 3 cm) was vertically compressed between the Recticel Pottscorer 410 and Recticel Bulpren S28280 foams on the pin plate and the second parallel plate. The hair was swiped twice in between the compressed foam such that the whole length of the hair bundle took three seconds to pass through. This was repeated on a different second and a third hair bundle. Each experiment as described above was repeated three times. The weights of the hair bundles were recorded and the results calculated as grams of Carbopol 956 solution deposited per gram of hair.

### 9. EXAMPLES

Several exemplary compositions are described below for incorporation within one or more exemplary absorbent substrates. The absorbent substrates of the following examples can be optionally adhered to a tool. Preferably, the absorbent substrates can be mounted onto two injection moulded polypropylene plates of 12.5 cm² surface area, hinged together, with the absorbent substrates facing one another. The angle between the plates is about 50°.

### Example A

### Hair Highlighting Using Peroxide and Alkalizer Gels Via an Absorbent Substrate

Two 12.5 cm² circular disks are cut of about 150 grams-per-square meter polyester high loft batting non-woven substrate (01-766 DI-8 available from Libeltex, Belgium; FB-215 available from PGI, New Jersey). Also polyurethane sponge (Bulpren S28280 available from Recticel International, Belgium) may be employed within this example.

| Peroxide Gel (1) | % w/w | Alkalizer Gel (2) | % w/w |
|---|---|---|---|
| De-ionized Water | q.s. to | De-ionized Water | q.s. to |
| | 100% | | 100% |
| Glycerine | 5.00 | Ammonium Hydroxide | 0.00 |
| | | (30% Active) | |
| Hydrogen Peroxide | 17.20 | Ammonium bicarbonate | 20.0 |
| (35% Active) | | (100% active) | |
| Disodium EDTA | 0.04 | Carbopol 956 | 1.80 |
| Carbopol 956 | 2.25 | pH | 8.80 |
| Sodium Hydroxide. | q.s. to | | |
| (50% aq Solution) | pH 2.9 | | |
| Viscosity | 15,400 cPs | Viscosity | 8,100 cPs |

Viscosity of gels 1 and 2 when mixed = 24,000 cPs

The Carbopol 956 used to prepare the peroxide gel is hydrated in rapidly mixing water until homogenous either by slow manual addition or by using an eductor or similar device for rapid hydration of powders. This example specifically reports Carbopol 956 as the thickening agent for the peroxide and alkalizer gels, but other thickening agents are contemplated. The hydrogen peroxide is then added with moderate mixing so as not to introduce excess air bubbles into the system. Then, 50% sodium hydroxide is added dropwise as appropriate to adjust the pH to the indicated value. Optionally, additional peroxide stabilizers such as sodium stannate may be added to further reduce the likelihood of premature peroxide decomposition. Once the peroxide gel is prepared the viscosity according to the test method described herein above is measured.

The alkalizer gel is produced by hydrating the Carbopol 956 in rapidly mixing water either by slow manual addition or by using an eductor or similar device for rapid hydration of powders. When the Carbopol 956 is fully dispersed and homogenous, the ammonium hydroxide or ammonium bicarbonate is added with moderate mixing so as to avoid entrapping excess air bubbles. The batch will thicken and clear with the addition of the alkalizer. Once the alkalizer gel is prepared the viscosity according to the test method described herein above is measured.

Approximately 4 grams of peroxide gel 1 are loaded into one of the two absorbent substrate disks and approximately 4 grams of the alkalizer gel 2 are loaded into the other absorbent substrate disk. The gels are loaded by even application across the absorbent substrate surface with a pipette or syringe with gentle mechanical pressing with the pipette or syringe to ensure that the majority of the fluid is fully absorbed into the substrate and without sacrificing the integrity of the absorbent substrate.

The plates are pressed together from two to ten times so to compress and squeeze the absorbent substrates and to sufficiently mix the peroxide gel and the alkalizer gel.

Once the hair treatment application system is ready it is used to treat the hair according to the hair treatment test described below. The hair so treated has experienced bleaching compared to a control hair tress as determined on a Minolta Spectrograph.

### Hair Treatment Test

A 30.5 cm 0.8 gram tress of human hair (Caucasion Light Brown—International Hair Imports and Products, Valhalla, N.Y.) is prepared by binding one end of the hair strands with a plastic cable tie about 2 cm from the hair ends and further securing with a 3 cm strip of electrician's tape. This hair tress is hung on a stainless-steel slotted holder. The top of the hair tress is then contacted with the hair treatment application system simulating the root-line on a real human head. The hair treatment application system is then pulled through to the end of the hair tress while maintaining the force with the user's fingers. The application may be repeated a second time. The resulting hair tress, which has between 0.3 to 0.8 grams of the hair treatment compositions deposited onto it, is then placed on a weigh boat and stored in an oven at 30° C. for 30 minutes. The hair tress is then rinsed with water and left to dry.

### Example B

### Hair Coloured Highlights Using Peroxide and Oxidative Dyes Gels Via an Absorbent Substrate

Absorbent substrates are prepared as described in Example A above.

| | | Oxidative Dyes + | |
|---|---|---|---|
| Peroxide Gel (3) | % w/w | Alkalizer Gel (4) | % w/w |
| De-ionized Water | q.s. to 100% | De-ionized Water | q.s. to |
| | | | 100 % |
| Glycerine | 5.00 | Ethanolamine | 4.00 |
| Hydrogen Peroxide | 17.20 | Propylene glycol | 5.00 |
| (35% Active) | | | |
| Disodium EDTA | 0.04 | Carbopol 956 | 1.00 |
| Carbopol 956 | 1.00 | Glycerine | 5.00 |
| Sodium Hydroxide | q.s. to | Sodium Sulphite | 0.30 |
| (50% aq. olution) | pH 3.5 | | |
| Viscosity | 10,500 cPs | EDTA | 0.10 |
| | | Erythorbic acid | 0.40 |
| | | 1-Naphthol | 0.10 |
| | | Para-aminophenol | 0.85 |
| | | 1-hydroxy-4,5- | 0.30 |
| | | diaminopyrazole sulfate | |
| | | Phenyl Methyl | 0.20 |
| | | Pyrazolone | |
| | | 2-methyl-5-hydroxyethyl | 1.50 |
| | | aminophenol | |
| | | pH | 10.0 |
| | | Viscosity | 6,800 cPs |

Viscosity of gels 3 and 4 when mixed = 17,100 cPs

The peroxide gel 3 is produced by combining the Carbopol 956 with the glycerine and mixing until homogenous slurry is obtained. De-ionized water is charged into a separate container of sufficient size to contain the entire batch. The slurry is introduced into the water slowly and mixed with moderate agitation until a stable, homogenous gel is observed. The hydrogen peroxide is then added with moderate mixing so as not to introduce excess air bubbles into the system. Then, sodium hydroxide is added dropwise to increase the pH to 3.5. Optionally, additional peroxide stabilizers such as sodium stannate may be added to further reduce the likelihood of premature peroxide decomposition.

Oxidative dye and alkalizer gel 4 is produced by hydrating the Carbopol 956 in rapidly mixing water either by slow manual addition or by using an eductor or similar device for rapid hydration of powders. When the Carbopol 956 is fully dispersed and homogenous, all the remaining ingredients are added, apart from the ethanolamine (i.e. glycerine, dye precursors, pH buffers and antioxidants). Once they have dissolved, the ethanolamine is added with moderate mixing so as to avoid entrapping excess air bubbles. The batch will thicken and clear with the addition of the alkalizer. A hair tress is treated as described above in the hair treatment test. A light copper shade was determined on a Minolta Spectograph on the treated tress when compared to a control hair tress.

### Example C

### Hair Highlights Using Persulfates and Peroxide Gel Via an Absorbent Substrate

Absorbent substrates are prepared as described in Example A above.

Approximately 6 grams of peroxide gel 3 are mixed with 4 grams of persulfate powder 5 in a weigh boat with a spatula. Approximately 4 grams of the resulting mixture are loaded into each of the absorbent substrates and a hair tress is treated as described above in the hair treatment test.

| Peroxide Gel (3) | % w/w | Persulfate powder (5) | % w/w |
|---|---|---|---|
| De-ionized Water | q.s. to 100% | Ammonium Persulfate | 28.60 |
| Glycerine | 5.00 | Potassium Persulfate | 50.00 |
| Hydrogen Peroxide | 17.20 | Sodium Persulfate | 7.14 |
| (35% Active) | | | |
| Disodium EDTA | 0.04 | Sodium Metasilicate | 14.29 |
| Carbopol 956 | 1.00 | | |
| Sodium Hydroxide | q.s. to pH 3.5 | | |
| (50% aq. Solution) | | | |
| Viscosity | 10,500 cPs | | |

Viscosity of gels 3 and 5 when mixed = 6,000 cPs

Peroxide gel 3 is prepared as described above in example B. Persulfate powders are produced by the dry blending all the ingredients, in any order, in a suitable blending apparatus such as a V-blender. The composition should be combined to homogeneity. The persulfate powder so formed is then pre-mixed with the peroxide gel. The mixing may encompass a bottle, bowl or tray where the ingredients are intermixed together via shaking or stirring, or it can involve a dual dispensing device which mixes the components as they are being added to the device. The resulting composition is loaded into the absorbent substrate and then applied to the hair.

This hair highlighting composition may provide a high level of decolorizing effect in a short amount of time. The resulting hair tress has experienced significant bleaching compared to a control hair tress as determined on a Minolta Spectograph.

### Example D

### Root-Touch-Up Using Direct Dye Gel or Peroxide Gel with Oxidative Dyes Via an Absorbent Substrate

A 5×5 cm square piece (25 cm²) is cut from a roll of about 150 grams-per-square meter polyester high loft batt non-woven substrate (01-766 DI-8 available from Libeltex, Belgium). The absorbent piece is adhered to the center of a 6×6 cm piece of 1 mm thick clear polyethylene film with 3M two-sided tape. Approximately 7.5 grams of direct dye gel 6 is loaded into the absorbent substrate (3,000 grams of direct dye gel 6 per square meter of absorbent substrate) on a weigh scale using a pipette or a syringe. The direct dye gel 6 is applied evenly across the absorbent substrate surface with gentle mechanical pressing with the pipette or syringe to ensure that the majority of the direct dye gel 6 is fully absorbed into the substrate. The resulting loaded absorbent substrate adhered to the polyethylene film is then rubbed into the root-line of a mannequin head (available from Salons Direct (Pro-hair)) with gloved fingers touching the polyethylene side and the absorbent substrate side being rubbed into and across the hair root-line. The resulting mannequin head is left to sit for 30 minutes and then rinsed and shampooed. The root-line of the mannequin head has experienced a light brown coloration.

| Direct Dyes Gel (6) | % w/w |
|---|---|
| De-ionized Water | q.s. to 100% |
| HC Yellow No. 2 | 0.20 |
| Disperse Black 9 | 0.05 |
| HC Red No. 3 | 0.15 |
| Disperse Violet 1 | 0.05 |
| Erythorbic Acid | 0.025 |
| Citric Acid | 0.5 |
| Ethanolamine | 2.5 |
| Carbopol 956 | 0.83 |
| HC Orange No. 1 | 0.1 |
| pH | 10.1 |
| Viscosity | 12,100 cPs |

Root-touch-up can be achieved with oxidative dyes by employing the exact procedure above, but with pre-mixing 4 grams of peroxide gel 3 and 4 grams of oxidative dye and alkalizer gel 7 as described below. An amount of 7.5 grams of resulting mixture is loaded into the absorbent substrate as described above.

| | | Oxidative Dyes + | |
|---|---|---|---|
| Peroxide Gel (3) | % w/w | Alkalizer Gel (7) | % w/w |
| De-ionized Water | q.s. to 100% | De-ionized Water | q.s. to 100% |
| Glycerine | 5.00 | Ethanolamine | 4.00 |
| Hydrogen Peroxide | 17.20 | Propylene glycol | 5.00 |
| (35% Active) | | | |
| Disodium EDTA | 0.04 | Carbopol 956 | 1.00 |
| Carbopol 956 | 1.00 | Glycerine | 5.00 |
| Sodium Hydroxide | q.s. to | Sodium Sulphite | 0.30 |
| (50% aq. Solution) | pH 3.5 | | |
| Viscosity | 10,500 cPs | EDTA | 0.10 |
| | | Erythorbic acid | 0.40 |
| | | Meta-aminophenol | 0.10 |
| | | Para- | 0.30 |
| | | phenylenediamine | |
| | | N,N-Bis(2- | 0.03 |
| | | Hydroxyethyl)-P- | |
| | | Phenylenediamine | |
| | | Resorcinol | 0.30 |
| | | Phenyl Methyl | 0.10 |
| | | Pyrazolone | |
| | | Viscosity | 13,400 cPs |

Viscosity of gels 3 and 7 when mixed = 20,700 cPs

Oxidative dye and alkalizer gel 7 is produced by hydrating the Carbopol 956 in rapidly mixing water either by slow manual addition (so as not to produce “fisheyes” of undispersed polymer) or by using an eductor or similar device for rapid hydration of powders. When the Carbopol 956 is fully dispersed and homogenous, the remaining ingredients are added, apart from the ethanolamine (i.e. glycerine, dye precursors, pH buffers and antioxidants). Once they have dissolved, the ethanolamine is added with moderate mixing so as to avoid entrapping excess air bubbles. The batch will thicken and clear with the addition of the alkalizer. The peroxide gel 3 and the oxidative dyes and alkalizer gel 7 are mixed and the resulting composition is applied on a mannequin head as described above at the root-line. After removing the composition a light brown coloration has been observed.

The absorbent substrates according to the present invention may also be used to broadly treat substrates such as hair, teeth, finger nails, textiles, and animal fur. Non-limiting additional examples of other hair treatment compositions and cosmetic compositions having viscosity as selected herein are exemplified here below.

| Example E. Shampoo composition | % w/w |
|---|---|
| De-ionized Water | q.s. to 100% |
| Sodium laureth sulfate | 12.0 |
| Cocamidopropyl betaine | 3.0 |
| Viscosity | 3,750 cPs |

| Example F. Conditioner shampoos | % w/w |
|---|---|
| De-ionized Water | q.s. to 100% |
| Ammonium Laureth Sulfate | 10.0000 |
| Ammonium Lauryl Sulfate | 6.0000 |
| Cocamidopropyl Betaine | |
| Sodium Lauroamphoacetate | |
| Cocamide MEA | 0.8000 |
| Cetyl Alcohol | 0.9000 |
| Ethylene Glycol Distearate | 1.5000 |
| Dimethicone Viscasil 330,000 | 1.3500 |
| Dow Corning 1664 300 nm/60M emulsion | |
| Polyquaternium-10 (LR30M) | 0.5000 |
| Polyox PEG7M | 0.1000 |
| Puresyn 6 (1-decene homopolymer) | 0.3000 |
| Perfume | 0.5000 |
| Citric Acid | 0.0400 |
| Sodium Citrate Dihydrate | 0.3972 |
| Disodium EDTA | 0.0993 |
| Kathon | 0.0005 |
| Sodium Benzoate | 0.2500 |
| Sodium Chloride | 0–3 |
| Ammonium Xylene Sulfonate | 0–3 |
| Viscosity | 8,000 cPs |
| De-ionized Water | q.s. to 100% |
| Cetyl Alcohol *1 | 0.96 |
| Stearyl Alcohol *2 | 0.64 |
| Stearamidopropyl Dimethylamine *3 | 1.0 |
| Zinc pyrithione *4 | 2.0 |
| Benzyl alcohol | 0.4 |
| Phenoxy Ethanol | 0.3 |
| Methyl Paraben | 0.2 |
| Propyl Paraben | 0.1 |
| Hydroxyethyl Cellulose | 0.25 |
| PEG-2M | 0.5 |
| Emulsifying Wax | 0.5 |
| Perfume | 0.4 |
| Citric acid - (adjust to pH 3–7) | 0.4 |
| Viscosity | 11,500 cPs |

*1 Cetyl Alcohol: Konol series available from Shin Nihon Rika.
*2 Stearyl Alcohol: Konol series available from Shin Nihon Rika.
*3 Stearamidopropyl Dimethylamine: SAPDMA available from Inolex.
*4 Zinc pyrithinone: Zinc pyrithione U/2 available from Olin

| Example H. Hair Styling Gels | % w/w |
|---|---|
| De-ionized Water | q.s. to 100% |
| Acrylates/beheneth-25 Methylacrylate Copol | 2.50 |
| NATRASOL 250 | 0.558 |
| Benzyl alcohol | 0.5 |
| Acrylates Copolymer | 0.4 |
| Polyquaternium 4 | 1.40 |
| Laureth 23 (Polyoxyethylene (23) Lauryl+ | 1.60 |
| Viscosity | 57,500 cPs |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed a “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A hair treatment application system comprising
at least one absorbent substrate and
one or more hair treatment compositions,
wherein said at least one absorbent substrate has a median pore radius of from about 300 microns to about 3,000 microns and
wherein said one or more hair treatment compositions each has a viscosity of from about 3,000 to about 150,000 cps.

2. A hair treatment application system according to claim 1, wherein said viscosity is measured before said one or more hair treatment compositions are applied to said at least one absorbent substrate.

3. A hair treatment application system according to claim 1, wherein said at least one absorbent substrate has a median pore radius of from about 400 microns to about 2,500 microns and wherein said one or more hair treatment compositions each has a viscosity of from about 5,000 cps to about 125,000 cps.

4. A hair treatment application system according to claim 1, wherein said at least one absorbent substrate has a median pore radius of from about 450 microns to about 2,000 microns and wherein said one or more hair treatment compositions each has a viscosity of about 7,000 cps to about 100,000 cps.

5. A hair treatment application system according to claim 1, wherein said at least one absorbent substrate has a median pore radius of from about 500 microns to about 1,800 microns and wherein said one or more hair treatment compositions each has a viscosity of from about 9,000 cps to about 85,000 cps.

6. A hair treatment application system according to claim 1, wherein said at least one absorbent substrate is selected from non-wovens and porous foams.

7. A hair treatment application system according to claim 1, wherein said hair treatment application system comprises a first and a second absorbent substrate and wherein said first absorbent substrate is for comprising a first hair treatment composition and wherein said second absorbent substrate is for comprising a second hair treatment composition.

8. A hair treatment application system according to claim 1, wherein said hair treatment application system further comprises an application tool.

9. A hair treatment application system according to claim 8, wherein said application tool comprises at least one plate.

10. A hair treatment application system according to claim 8, wherein said application tool comprises two plates and wherein at least one absorbent substrate is attached to at least one of the two plates.

11. A hair treatment application system according to claim 10, wherein said two plates are two interconnected plates.

12. A hair treatment application system according to claim 10, wherein said two plates are connected via a hinge.

13. A hair treatment application system according to claim 1 wherein said at least one absorbent substrate has an absorption capacity of from about 10 to about 80 grams of liquid per gram of absorbent substrate.

14. A hair treatment application system according to claim 13, wherein said one or more hair treatment compositions each has a viscosity of from about 5,000 to about 125,000 cps and wherein said at least one absorbent substrate has a median pore radius of from about 400 microns to about 2,500 microns and an absorption capacity of from about 15 to about 75 grams of liquid per gram of substrate.

15. A hair treatment application system according to claim 13, wherein said one or more hair treatment compositions each has a viscosity of from about 7,000 to about 100,000 cps and wherein said at least one absorbent substrate has a median pore radius of from about 450 microns to about 2,000 microns and an absorption capacity of from about 20 to about 70 grams of liquid per gram of absorbent substrate.

16. A hair treatment application system according to claim 13, wherein said one or more hair treatment compositions each has a viscosity of from about 9,000 to about 85,000 cps and wherein said at least one absorbent substrate has a median pore radius of from about 500 microns to about 1,800 microns and an absorption capacity of from about 25 to about 65 grams of liquid per gram of absorbent substrate.

17. A hair treatment application system according to claim 13, wherein said at least one absorbent substrate is selected from non-wovens and porous foams.

18. A hair treatment application system according to claim 13, wherein said hair treatment application system comprises a first and a second absorbent substrate and wherein said first absorbent substrate is for comprising a first hair treatment composition and wherein said second absorbent substrate is for comprising a second hair treatment composition.

19. A hair treatment application system according to claim 13, wherein said hair treatment application system further comprises an application tool.

20. A hair treatment application system according to claim 19, wherein said application tool comprises at least one plate.

21. A hair treatment application system according to claim 19, wherein said application tool comprises two plates, wherein at least one absorbent substrate is attached to at least one of the two plates.

22. A hair treatment application system according to claim 21, wherein said two plates are interconnected.

23. A hair treatment application system according to claim 21, wherein said two plates are connected via a hinge.

24. A method to treat the hair comprising the step of contacting the hair with a hair treatment application system according to claim 1.

25. A method to treat the hair comprising the step of contacting the hair with a hair treatment application system according to claim 12.

26. A method according to claim 24, wherein said one or more hair treatment compositions are loaded into said at least one absorbent substrate before contacting the hair with said hair treatment application system.

27. A method according to claim 25, wherein said one or more hair treatment compositions are loaded into said at least one absorbent substrate before contacting the hair with said hair treatment application system.

28. A method according to claim 24, wherein said one or more hair treatment compositions comprises at least two hair treatment compositions, and wherein the method comprises the step of mixing said at least two hair treatment compositions before loading the same into said at least one absorbent substrate.

29. A method according to claim 25, wherein said one or more hair treatment compositions comprises at least two hair treatment compositions, and wherein the method comprises the step of mixing said at least two hair treatment compositions before loading the same into said at least one absorbent substrate.

30. A method according to claim 28, wherein one of said at least two hair treatment compositions comprises an oxidizing agent and wherein another of said at least two hair treatment compositions comprises an alkalising agent.

31. A method according to claim 29, wherein one of said at least two hair treatment compositions comprises an oxidizing agent and wherein another of said at least two hair treatment compositions comprises an alkalising agent.

32. A method according to claim 24, wherein said one or more hair treatment compositions are selected from the group consisting of shampoo compositions, conditioning compositions, styling compositions, hair colourant compositions, hair bleaching compositions, highlighting compositions and combinations thereof.

33. A method according to claim 25, wherein said one or more hair treatment compositions are selected from the group consisting of shampoo compositions, conditioning compositions, styling compositions, hair colourant compositions, hair bleaching compositions, highlighting compositions and combinations thereof.

34. A method according to claim 24, wherein said hair treatment application system comprises a first and a second absorbent substrate; wherein said first absorbent substrate comprises a first hair treatment composition and said second absorbent substrate comprises a second hair treatment composition; and wherein the method comprises the step of mixing said first and second hair treatment compositions by squeezing together at least once said first and second absorbent substrates before contacting the hair with the hair treatment application system.

35. A method according to claim 25, wherein said hair treatment application system comprises a first and a second absorbent substrate; wherein said first absorbent substrate comprises a first hair treatment composition and said second absorbent substrate comprises a second hair treatment composition; and wherein the method comprises the step of mixing said first and second hair treatment compositions by squeezing together at least once said first and second absorbent substrates before contacting the hair with the hair treatment application system.

36. A method according to claim 34, wherein said wherein said first hair treatment composition comprises an oxidizing agent and said second hair treatment composition comprises an alkalising agent.

37. A method according to claim 35, wherein said first hair treatment composition comprises an oxidizing agent and said second hair treatment composition comprises an alkalising agent.

38. A method according to claim 34, wherein said step of mixing said first and second hair treatment compositions comprises squeezing together more than once said first and second absorbent substrates before contacting the hair with the hair treatment application system.

39. A method according to claim 35, wherein said step of mixing said first and second hair treatment compositions comprises squeezing together more than once said first and second absorbent substrates before contacting the hair with the hair treatment application system.

40. A method according to claim 38, wherein said first hair treatment composition comprises an oxidizing agent and said second hair treatment composition comprises an alkalising agent.

41. A method according to claim 39, wherein said first hair treatment composition comprises an oxidizing agent and said second hair treatment composition comprises an alkalising agent.

42. A kit-of-parts comprising a hair treatment application system according to claim 1.

43. A kit-of-parts comprising a hair treatment application system according to claim 13.

44. A kit-of-parts according to claim 42, wherein said kit further comprises
a. an individually packaged composition comprising an oxidizing agent and
b. an individually packaged composition comprising an alkalizing agent.

45. A kit-of-parts according to claim 43, wherein said kit further comprises
a. an individually packaged composition comprising an oxidizing agent and
b. an individually packaged composition comprising an alkalizing agent.

46. A kit-of-parts according to claim 42, wherein kit further comprises
a. an individually packaged composition comprising an oxidizing agent and
b. an individually packaged composition comprising an alkalizing agent;
wherein said individually packaged composition comprising an alkalizing agent alkalizing agent comprises a persulfate salt.

47. A kit-of-parts according to claim 43, wherein kit further comprises
c. an individually packaged composition comprising an oxidizing agent and
d. an individually packaged composition comprising an alkalizing agent;
wherein said individually packaged composition comprising an alkalizing agent alkalizing agent comprises a persulfate salt.

48. A kit-of-parts according to claim 42, wherein said kit further comprises at least one additional individually packaged compositions selected from the group consisting of shampoo compositions, conditioning compositions, styling compositions, hair colourant compositions, hair bleaching compositions, highlighting compositions and combinations thereof.

49. A kit-of-parts according to claim 43, wherein said kit further comprises at least one additional individually packaged compositions selected from the group consisting of shampoo compositions, conditioning compositions, styling compositions, hair colourant compositions, hair bleaching compositions, highlighting compositions and combinations thereof.

50. A kit-of-parts according to claim 42, wherein said kit further comprises means to mix and/or load said individually packaged compositions into said at least one absorbent substrate.

51. A kit-of-parts according to claim 43, wherein said kit further comprises means to mix and/or load said individually packaged compositions into said at least one absorbent substrate.

52. A kit-of-parts according to claim 42, wherein said kit of part further comprises instruction for the use of said hair treatment application system.

53. A kit-of-parts according to claim 43, wherein said kit of part further comprises instruction for the use of said hair treatment application system.
